(19) United States
(12) Patent Application Publication
(10) Pub. No.: US 2002/0169561 A1
(43) Pub. Date: Nov. 14, 2002

(54) Modular computational models for predicting the pharmaceutical properties of chemical compunds

(76) Inventors: Albert S. Benight, Schaumburg, IL (US); Peter V. Riccelli, Tinley Park, IL (US); Anton J. Hopfinger, Lake Forest, IL (US); Petr Pancoska, Evanston, IL (US)

Correspondence Address: LOUIS MYERS, Fish & Richardson P.C., 225 Franklin Street, Boston, MA, 02110-2804, US

(21) Appl. No.: 10/058,655

(22) Filed: Jan. 28, 2002

Related U.S. Application Data
(60) Provisional application No. 60/264,640, filed on Jan. 26, 2001.

(51) Int. Cl. G06G 7/48; G06G 7/58; G06F 19/00; G01N 33/48; G01N 33/50

(52) U.S. Cl. 702/19; 703/11

(57) ABSTRACT

The methods of the invention allow for the construction and/or use of modular computational models to accurately predict the therapeutic properties, including both therapeutic potency and one or more ADMET properties, of all or part of a chemical compound. The modular computational models can be used to rapidly screen libraries of chemical compounds, and reliably identify small subsets of those chemical compounds that have desirable therapeutic potency and ADMET properties, and are thus the best overall drug candidates.

### RELATED APPLICATIONS

This application claims priority to U.S. provisional application No. 60/264,640, filed on Jan. 26, 2001, the contents of which are incorporated herein by reference.

### TECHNICAL FIELD

This invention relates to the generation of modular computer-based models that correlate the structure of a chemical compound with an activity, and the use of such models to screen libraries of chemical compounds and thereby reliably identify the best candidate compounds potentially having a desirable activity, e.g., a desirable pharmaceutical activity.

### BACKGROUND

Successful drug-candidate ligands typically bind to their therapeutic target receptors with high affinity. To be truly successful, however, drug-candidate ligands must also possess desirable ADMET (absorption, distribution, metabolism, excretion and toxicological) properties. The combination of high affinity receptor binding and proper ADMET properties controls the optimal expression of therapeutic biopotency and minimizes the side effects associated with administering a therapeutic drug to a patient.

Traditionally, drug candidates were identified through a time-consuming process of individually assaying the activity, e.g., receptor affinity, of each compound in a large library of compounds. After drug candidates were identified through this screening process, they would undergo further screening involving assays designed to assess their ADMET properties. Because of the time and resources required for such screens, there has been a growing effort to develop computational models for predicting, in the absence of experimental data about more than a fraction of compounds, whether an experimentally untested compound will bind to a receptor, and thus constitute a drug candidate. Similarly, there has been a movement to develop computational models that can predict the outcome of assays designed to test the ADMET properties of drug candidates. There remains in the art, however, a need to develop improved computational methods that more accurately predict the activity of compounds, with respect to both receptor affinity and ADMET properties. Such computational methods can be used to rapidly screen libraries of virtual compounds and identify drug candidates.

### SUMMARY

The methods of the invention allow for the construction and/or use of modular computational models to accurately predict one or more therapeutic properties, including therapeutic potency (e.g., receptor affinity) and ADMET (e.g., absorption, distribution, metabolism, excretion and toxicity) properties, of all or part of a chemical compound, e.g., a small molecule, protein (e.g., a peptide or modified peptide), or nucleic acid molecule. Preferably, the modular computational models are used to rapidly screen libraries of chemical compounds, thereby reliably identifying small subsets of those chemical compounds that are the best overall drug candidates.

Accordingly, in one aspect, the invention features methods of constructing a modular computational model for predicting one or more therapeutic properties, e.g., therapeutic potency (e.g., receptor affinity) or an ADMET property (e.g., absorption, distribution, metabolism, excretion and toxicity), of a chemical compound, e.g., a small molecule, protein (e.g., peptide or modified peptide), or nucleic acid molecule. The methods include:

obtaining a first set of data, e.g., composed of thermodynamic, spectroscopic, chromatographic, or biological (e.g., from a cell-based or animal-based assay) measurements, describing the interaction between each training compound of a first set of training compounds, e.g., small molecules, proteins (e.g., peptides or modified peptides), or nucleic acid molecules, and a first interaction partner, e.g., a molecule (e.g., a protein, lipid, or nucleic acid molecule), a supramolecular structure (e.g., a protein complex, lipid monolayer, lipid bilayer, a protein-nucleic acid complex, or any combination thereof), a cell, or a chromatographic column;

using the first set of data, along with data about the chemical structures, e.g., three dimensional atomic structures, and/or physical properties thereof, e.g., conformational freedom, hydrophobicity, dipole moment, solubility, electrostatic potential, permeability and, more generally, any property that can be derived from the chemical structure of a molecule, of the first set of training compounds and, optionally, data about the three dimensional structure and/or physical properties thereof of the first interaction partner, to construct a first module that uses data about the chemical structures and/or physical properties thereof of chemical compounds to predict values, e.g., thermodynamic, spectroscopic, chromatographic, or biological (e.g., from a cell-based or animal-based assay) values similar in type to those of the first set of data, describing the interaction between a chemical compound, e.g., a compound of the first set of training compounds or a member from a plurality of test structures (e.g., compounds that are structurally of functionally related to one or more compounds of the first set of training compounds), and the first interaction partner;

thereby constructing a single module modular computational model, consisting of a first module, for predicting one or more therapeutic properties, e.g., therapeutic potency (e.g., receptor affinity) or an ADMET property (e.g., absorption, distribution, metabolism, excretion and toxicity), of a chemical compound.

In preferred embodiments, the first set of data, e.g., thermodynamic, spectroscopic, chromatographic, or biological (e.g., from a cell-based or animal-based assay) measurements, is obtained experimentally as part of the methods of the invention. In other embodiments, the first set of data, e.g., thermodynamic, spectroscopic, chromatographic, or biological (e.g., from a cell-based or animal-based assay) measurements, are obtained from existing information sources, e.g., databases, scientific publications, or internet webpages. In other embodiments, the first set of data, e.g., thermodynamic, spectroscopic, chromatographic, or biological (e.g., from a cell-based or animal-based assay), is obtained, in part, experimentally as part of the methods of the invention and, in part, from existing information sources.

In some embodiments, the first set of data consists of, or is derived from, thermodynamic measurements, e.g., measurements of ΔH, ΔG, ΔS, equilibrium binding constants, ΔCp, and/or ΔV. Preferably, the thermodynamic measurements include a measurement of the enthalpy, ΔH. In other embodiments, the first set of data consists of, or is derived from, spectroscopic measurements, e.g., measurements of electromagnetic absorbance (e.g., ultraviolet, visible, or infrared light absorbance or circular dichroism), electromagnetic emission (e.g., fluorescence or nuclear magnetic resonance (NMR)), surface plasmon resonance, or mass spectroscopy. In other embodiments, the first set of data consists of, or is derived from, diffusion rate measurements or solubility measurements, e.g., measurements of the rate of diffusion or solubility in an aqueous medium. In still other embodiments, the first set of data consists of, or is derived from, cell-based or animal-based assay measurements, e.g., measurements of cellular permeability or toxicity, measurements of bioconversion (e.g., breakdown or modification of a chemical compound), measures of distribution and dynamics of a compound in a living system, or measurements of other cellular processes (e.g., inflammation).

In some embodiments, the first set of data consists of thermodynamic measurements made, e.g., using a calorimeter, such as a differential scanning calorimeter or an isothermal titration calorimeter. In preferred embodiments, at least some of the thermodynamic measurements are obtained in parallel, e.g., using a multi-cell calorimeter. In particularly preferred embodiments, at least some of the thermodynamic measurements are obtained in parallel using a multi-cell differential scanning calorimeter.

In other embodiments, the first set of data consists of spectroscopic measurements obtained, e.g., using a spectrophotometer (e.g., an ultraviolet, visible, or infrared spectrophotometer), a spectropolorimeter, a fluorimeter, an NMR detection instrument, a surface plasmon resonance instrument, or a mass spectroscopy instrument. In preferred embodiments, at least some of the spectroscopic measurements are obtained in parallel, e.g., using a mulit-cell or multi-cannel instrument, such as a multi-cell or multi-channel spectrophotometer, spectropolorimeter, fluorimeter, surface plasmon resonance instrument, or mass spectroscopy instrument.

In other embodiments, the first set of data consists of diffusion rate or solubility measurements obtained, e.g., using column chromatography (e.g., involving a hydrophobic, anion-exchange, cation-exchange, or size exclusion column mounted on, e.g., an HPLC instrument), a diffusion barrier instrument, a solubility instrument, or a capillary electrophoresis instrument. In preferred embodiments, at least some of the diffusion rate or solubility measurements are obtained in parallel, e.g., using a multi-cell or multi-channel instrument, such as a multi-cell or multi-channel column chromatography instrument, diffusion barrier instrument, solubility instrument, or capillary electrophoresis instrument.

In still other embodiments, the first set of data consists of biological (e.g., cell-based or animal-based assay) measurements obtained, e.g., using a visual imaging device (e.g., for counting cells, e.g., stained cells), a spectrophotometer, a spectropolorimeter, a fluorimeter, or a calorimeter. In preferred embodiments, at least some of the biological measurements are obtained in parallel, e.g., using a using a multi-cell or multi-cannel instrument, or an automated device, e.g., an automated imaging device.

In some embodiments, the first set of data, e.g., thermodynamic, spectroscopic, chromatographic, or biological (e.g., from a cell-based or animal-based assay) measurements, includes a single measurement for each compound in the first set of training compounds. In preferred embodiments, the first set of data includes a plurality of measurements, e.g., 2, 3, 4, 5, or more measurements, for each compound in the first set of training compounds.

In some embodiments, the first set of data, e.g., thermodynamic, spectroscopic, chromatographic, or biological (e.g., from a cell-based or animal-based assay) measurements, provides information relevant to therapeutic potency, e.g., binding affinity, of a chemical compound, e.g., a small molecule, protein (e.g., a peptide or modified peptide), or nucleic acid molecule, with respect to an interaction partner, e.g., a molecule (e.g., a protein, lipid, or nucleic acid molecule), a supramolecular structure (e.g., a protein complex, lipid monolayer, lipid bilayer, an in vitro or in vivo membrane system, a protein-nucleic acid complex, or any combination thereof), or a cell. In preferred embodiments, the measurements that provided information about therapeutic potency are thermodynamic measurements, e.g., measurements of ΔH, ΔG, ΔS, equilibrium binding constants, ΔCp, and/or ΔV. In preferred embodiments, the measurements that provide information about therapeutic potency include measurements of ΔH. In particularly preferred embodiments, the measurements that provide information about therapeutic potency include distinct measurements of ΔH, ΔG, and ΔS.

In other embodiments, the first set of data, e.g., thermodynamic, spectroscopic, chromatographic, or biological (e.g., from a cell-based or animal-based assay) measurements, provides information about one or more ADMET properties, e.g., absorption, distribution, metabolism, excretion, or toxicity, of a chemical compound, e.g., a small molecule, protein (e.g., a peptide or modified peptide), or nucleic acid molecule. In preferred embodiments, the ADMET property is absorption, e.g., as measured by permeability (e.g., cellular or membrane permeability), or toxicity, e.g., as measured by chemical conversion of the chemical compound or cellular toxicity in a cell-based or animal-based assay. In other preferred embodiments, the ADMET properties are absorption and distribution or active and passive diffusion, e.g., as measured by logP or permeability through in vitro or in vivo membrane systems.

In some embodiments, the values that provide information about one or more ADMET properties reflect the interaction of a chemical compound, e.g., a small molecule, protein (e.g., a peptide or modified peptide), or nucleic acid molecule, with an interaction partner, e.g., a molecule (e.g., a protein, lipid, or nucleic acid molecule), a supramolecular structure (e.g., a protein complex, lipid monolayer, lipid bilayer, an in vitro or in vivo membrane system, a protein-nucleic acid complex, or any combination thereof), a cell, or an animal. In other embodiments, the values that provide information about one or more ADMET properties reflect the interaction of a chemical compound, e.g., a small molecule, protein (e.g., a peptide or modified peptide), or nucleic acid molecule, with a solvent or a column (e.g., a hydrophobic, anion-exchange, cation-exchange, or size exclusion column or a capillary electrophoresis device).

In some embodiments, a compound of the first training set is a chemical compound, such as a small molecule, e.g., an organic compound, e.g., a fatty acid molecule, a sugar molecule, a steroid molecule, a hormone, a peptide, or any derivative or combination thereof. In other embodiments, a compound of the first training set is a chemical compound extracted from an animal, plant, fungus, or single cell organism, e.g., a bacterium or protist. In preferred embodiments, a compound of the first training set is a chemical compound that has been synthesized in a laboratory, e.g., by combinatorial chemistry or parallel synthesis.

In preferred embodiments, the first training set includes a plurality of training compounds, e.g., 5, 10, 20, 30, 40, 50, 75, 100, 125, 150, 200, or more training compounds.

In some embodiments, the interaction partner is a protein, e.g., a membrane associated protein (e.g., an adhesion receptor, a growth factor signaling receptor, a G-protein coupled receptor, a glycoprotein, or a transporter), a cytoplasmic protein (e.g., an enzyme, such as a carboxylase or transferase or ribosomal protein, a kinase, a phosphatase, an adapter molecule, a GTPase, or an ATPase), or a nuclear protein (e.g., a transcription factor, polymerase, or chromatin associated protein). In other embodiments, the interaction partner is a lipid, e.g., a modified lipid, e.g., phosphatidyl inositol 4, 5-phosphate or a similar lipid involved in signaling pathways. In other embodiments, the interaction partner is a nucleic acid molecule, e.g., DNA or RNA. In other embodiments, the interaction partner is a supramolecular structure, e.g., a multi-subunit protein complex, a protein-DNA or protein-RNA complex, a lipid membrane (e.g., a micelle, a lipid monolayer, or a lipid bilayer), or any combination thereof. In still other embodiments, the interaction partner is a cell, e.g., a mammalian cell, an insect cell, a fungal cell, a bacterium, or a protist.

In some embodiments, the interaction between one or more training compounds of the first set of training compounds and the first interaction partner includes, e.g., the formation of a chemical bond, e.g., a non-covalent bond (e.g., an ionic bond, van der Waals forces, or a combination thereof) or a covalent bond, between the training compound and the first interaction partner. In other embodiments, the interaction between one or more training compounds of the first set of training compounds and the first interaction partner includes, e.g., the breaking of a chemical bond, e.g., a non-covalent bond (e.g., an ionic bond, van der Waals forces, or a combination thereof) or a covalent bond, on either the training compound, the first interaction partner, or both. In other embodiments, the interaction between one or more training compounds of the first set of training compounds and the first interaction partner includes, e.g., the addition or removal of a chemical group, e.g., a phosphate group, on either the training compound, the first interaction partner, or both. In still other embodiments, the interaction between one or more training compounds of the first set of training compounds and the first interaction partner includes, e.g., the oxidation or reduction of a chemical group, e.g., an alcohol, ketone, or carboxylic acid group, on either the training compound, the first interaction partner, or both.

In preferred embodiments, the first set of data, e.g., thermodynamic, spectroscopic, chromatographic, or biological (e.g., from a cell-based or animal-based assay) measurements, is or was experimentally determined, e.g., by a method including the following steps:

providing, for each training compound of the first set of training compounds, at least one reaction mixture which optionally includes the first interaction partner;

inducing a change, e.g., a thermodynamic transition, in each reaction mixture; and

measuring, for each reaction mixture, the value of at least one parameter, e.g., a thermodynamic, spectroscopic, chromatographic, or biological (e.g., from a cell-based or animal-based assay) parameter, describing the interaction between a training compound and the first interaction partner.

In some embodiments, the change includes altering the concentration or activity of a training compound in the reaction mixture, e.g., via the addition of a training compound to each reaction mixture. In other embodiments, the change includes changing the concentration or activity of the first interaction partner, e.g., via the addition of the first interaction partner to each reaction mixture, or by contacting each reaction mixture with the first interaction partner. In other embodiments, the change includes changing the temperature of each reaction mixture.

In preferred embodiments, a plurality of, e.g., at least 5, 10, 20, 50, 100, 200, or more, measurements of a parameter, e.g., a thermodynamic, spectroscopic, chromatographic, or biological (e.g., from a cell-based or animal-based assay) parameter, are determined simultaneously, e.g., by using high throughput screening techniques, e.g., involving multi-cell or multi-channel instruments, e.g., multi-cell or multi-channel calorimeters, spectrophotometers, spectropolorimeters, fluorimeters, NMR detection instruments, mass spectroscopy, column chromatography instruments, diffusion barrier instruments, solubility instruments, capillary based techniques, microarrays or automated visual imaging devices.

In some embodiments, a plurality of, e.g., at least 5, 10, 20, 50, 100, 200, or more, training compounds from the first set of training compounds are determined simultaneously, e.g., in separate cells of a multicell or multi channel instrument. In other embodiments, a plurality of, e.g. at least 5, 10, 20, 50, or more, measurements of a parameter for a single training compound, e.g., under differing conditions, such as the concentration of the training compound or the interaction partner, or the temperature of the reaction mixture, are determined simultaneously.

In some embodiments, the data about the chemical structures and/or physical properties thereof for the first set of training compounds consists of the three dimensional atomic structures of each of the training compounds. In preferred embodiments, the data about the chemical structures and/or physical properties thereof for the first set of training compounds includes the three dimensional atomic structures of each of the training compounds, as well as information about the conformational freedom of the training compounds, e.g., a conformational ensemble profile. In other preferred embodiments, the data about the chemical structures and/or physical properties thereof for the first set of training compounds includes the three dimensional atomic structures of each of the training compounds, as well as information about relevant physical properties of the training compounds, such as hydrophobicity, dipole moment, solubility, electrostatic potential, permeability or, more generally, any property that can be derived from the chemical structure of a molecule. Relevant physical properties will depend upon the structures of the training compounds of the first set of training compounds and the therapeutic property or properties being predicted by the first module of the modular computational model. Such relevant physical properties can be determined as part of the process of constructing the first module of the modular computational model.

In some embodiments, data about the three-dimensional atomic structure and/or physical properties thereof of the interaction partner is included as part of the process of constructing the first module of the modular computational model. In some embodiments, the three-dimensional atomic structure of the interaction partner is well-defined, e.g., when the interaction partner is a protein, nucleic acid molecule, sugar chain, or any combination thereof, and the three-dimensional atomic structure of the interaction partner has been determined, e.g., using crystallography or multi-dimensional NMR. In other embodiments, the three-dimensional atomic structure of the interaction partner is only partially defined, e.g., when the interaction partner is a collection of lipid molecules, e.g., a micelle, a lipid monolayer, a lipid bilayer, or any membrane having characteristics identical to or consistent with a biological membrane. In some embodiments, data about the three-dimensional atomic structure and/or physical properties thereof of the interaction partner is not included as part of the process of constructing the first module of the modular computational model.

In preferred embodiments, the process of constructing the first module of the modular computational model includes techniques commonly used in the construction of quantitative structure-activity relationship (QSAR) models. In particularly preferred embodiments, the process of constructing the first module of the modular computational model includes techniques used in the construction of free energy force field QSAR (FEFF-QSAR) models, three-dimensional QSAR (3D-QSAR) models, four dimensional QSAR (4D-QSAR) models, or membrane interaction QSAR (MI-QSAR) models. In some embodiments, the process of constructing the first module of the modular computational model includes techniques commonly used in the construction of receptor dependent QSAR models, e.g., FEFF-QSAR models, receptor-dependent 4D-QSAR models, or MI-QSAR models. In other embodiments, the process of constructing the first module of the modular computational model includes techniques commonly used in the construction of receptor independent QSAR models, e.g., receptor independent 3D-QSAR models and receptor independent 4D-QSAR models.

In preferred embodiments, the process of constructing the first module of the modular computational model includes the use, e.g., at least once but preferably multiple times, of a partial least squares regression. For example, the partial least squares regression can be used to correlate the values of the first set of data with the data about the chemical structures and/or physical properties thereof of the compounds of the first set of training compounds. In other preferred embodiments, the process of constructing the first module of the modular computational model includes the use, e.g., at least once but preferably multiple times, of a genetic function algorithm (GFA). For example, the GFA can be used to identify features of the chemical structures, e.g., three-dimensional atom structures, and/or physical properties thereof, e.g., conformational freedom, hydrophobicity, dipole moment, solubility, etc., that correlate best with the values of the first set of data. In particularly preferred embodiments, the process of constructing the first module of the modular computational model includes the use, e.g., the alternating use, of both a partial least squares regression and a GFA.

In some embodiments, the first model can be refined, e.g., after being constructed, by the following method:

obtaining a supplemental first set of data, e.g., composed of data similar to the data of the first set of data, e.g., thermodynamic, spectroscopic, chromatographic, or biological (e.g., from a cell-based or animal-based assay), that describes the interaction between each training compound of a supplemental first set of training compounds, e.g., small molecules, proteins (e.g., peptides or modified peptides), or nucleic acid molecules, that are, e.g., structurally or functionally related to the compounds of the first set of training compounds, and the first interaction partner; and

using the first set of data and the supplemental first set of data, along with data about the chemical structures, e.g., three dimensional atomic structures, and/or physical properties thereof, e.g., conformational freedom, hydrophobicity, dipole moment, solubility, electrostatic potential, permeability and, more generally, any property that can be derived from the chemical structure of a molecule, of the first set of training compounds and the supplemental first set of training compounds, and, optionally, using data about the three dimensional structure and/or physical properties thereof of the first interaction partner, to reconstruct the first computational module, e.g., by the same process used to construct the first computational module;

thereby refining the first module of a modular computational model.

In some embodiments, the supplemental first set of training compounds, e.g., small molecules, proteins (e.g., peptides or modified peptides), or nucleic acid molecules, consists of compounds that are structurally or functionally related to the compounds of the first set of training compounds. In other embodiments, the supplemental first set of training compounds, e.g., small molecules, proteins (e.g., peptides or modified peptides), or nucleic acid molecules, consists of at least some compounds that are identical to some of the compounds of the first set of training molecules. For example, the supplemental first set of data could be obtained to either extend the first set of data, to verify some or all of the measurements of the first set of data, or both.

In preferred embodiments, the supplemental first set of data is obtained experimentally using the same experimental techniques used to produce the first set of data. In other embodiments, the supplemental first set of data is obtained experimentally using experimental techniques different from those used to produce the first set of data, e.g., the experimental techniques can be different approaches to measuring the same value, e.g., thermodynamic, spectroscopic, chromatographic, or biological (e.g., from a cell-based or animal-based assay) value. In some embodiments, the supplemental first set of data is obtained from existing information sources, e.g., databases, scientific publications, or internet webpages.

In preferred embodiments, a modular computational model of the invention includes, e.g., two, three, four, five, six, or more modules, constructed, e.g., by a process analogous to the process used to construct the first module of the modular computational model. Thus, the methods of constructing a modular computational model for predicting one or more therapeutic properties, e.g., therapeutic potency (e.g., receptor affinity) or an ADMET property (e.g., absorption, distribution, metabolism, excretion and toxicity), of a chemical compound, e.g., a small molecule, protein (e.g., peptide or modified peptide), or nucleic acid molecule can further include:

obtaining a second set of data, e.g., composed of thermodynamic, spectroscopic, chromatographic, or biological (e.g., from a cell-based or animal-based assay) measurements, describing the interaction between each training compound of a second set of training compounds, e.g., small molecules, proteins (e.g., peptides or modified peptides), or nucleic acid molecules, and a second interaction partner, e.g., a molecule (e.g., a protein, lipid, or nucleic acid molecule), a supramolecular structure (e.g., a protein complex, lipid monolayer, lipid bilayer, a protein-nucleic acid complex, or any combination thereof), a cell, or a chromatographic column;

using the second set of data, along with data about the chemical structures, e.g., three dimensional atomic structures, and/or physical properties thereof, e.g., conformational freedom, hydrophobicity, dipole moment, solubility, electrostatic potential, permeability and, more generally, any property that can be derived from the chemical structure of a molecule, of the second set of training compounds and, optionally, data about the three dimensional structure and/or physical properties thereof of the second interaction partner, to construct a second module that uses data about the chemical structures and/or physical properties thereof of chemical compounds to predict values, e.g., thermodynamic, spectroscopic, chromatographic, or biological (e.g., from a cell-based or animal-based assay) values similar in type to those of the second set of data, describing the interaction between a chemical compound, e.g., a compound of the second set of training compounds or a member from a plurality of test structures (e.g., compounds that are structurally of functionally related to one or more compounds of the second set of training compounds), and the second interaction partner;

thereby constructing a two module modular computational model, consisting of a first and a second module, for predicting one or more therapeutic properties, e.g., therapeutic potency (e.g., receptor affinity) or an ADMET property (e.g., absorption, distribution, metabolism, excretion and toxicity), of a chemical compound.

In preferred embodiments, the second set of data, e.g., thermodynamic, spectroscopic, chromatographic, or biological (e.g., from a cell-based or animal-based assay) measurements, is obtained experimentally as part of the methods of the invention. In other embodiments, the second set of data, e.g., thermodynamic, spectroscopic, chromatographic, or biological (e.g., from a cell-based or animal-based assay) measurements, are obtained from existing information sources, e.g., databases, scientific publications, or internet webpages. In other embodiments, the second set of data, e.g., thermodynamic, spectroscopic, chromatographic, or biological (e.g., from a cell-based or animal-based assay), is obtained, in part, experimentally as part of the methods of the invention and, in part, from existing information sources.

In some embodiments, the second set of data consists of, or is derived from, thermodynamic measurements, e.g., measurements of ΔH, ΔG, ΔS, equilibrium binding constants, ΔCp, and/or ΔV. Preferably, the thermodynamic measurements include a measurement of the enthalpy, ΔH. In other embodiments, the second set of data consists of, or is derived from, spectroscopic measurements, e.g., measurements of electromagnetic absorbance (e.g., ultraviolet, visible, or infrared light absorbance or circular dichroism), electromagnetic emission (e.g., fluorescence or nuclear magnetic resonance (NMR)), surface plasmon resonance, or mass spectroscopy. In other embodiments, the second set of data consists of, or is derived from, diffusion rate measurements or solubility measurements, e.g., measurements of the rate of diffusion or solubility in an aqueous medium. In still other embodiments, the second set of data consists of, or is derived from, cell-based or animal-based assay measurements, e.g., measurements of cellular permeability or toxicity, measurements of bioconversion (e.g., breakdown or modification of a chemical compound), measures of distribution and dynamics of a compound in a living system, or measurements of other cellular processes (e.g., inflammation).

In some embodiments, the second set of data consists of thermodynamic measurements made, e.g., using a calorimeter, such as a differential scanning calorimeter or an isothermal titration calorimeter. In preferred embodiments, at least some of the thermodynamic measurements are obtained in parallel, e.g., using a multi-cell calorimeter. In particularly preferred embodiments, at least some of the thermodynamic measurements are obtained in parallel using a multi-cell differential scanning calorimeter.

In other embodiments, the second set of data consists of spectroscopic measurements obtained, e.g., using a spectrophotometer (e.g., an ultraviolet, visible, or infrared spectrophotometer), a spectropolorimeter, a fluorimeter, an NMR detection instrument, a surface plasmon resonance instrument, or a mass spectroscopy instrument. In preferred embodiments, at least some of the spectroscopic measurements are obtained in parallel, e.g., using a mulit-cell or multi-cannel instrument, such as a multi-cell or multi-channel spectrophotometer, spectropolorimeter, fluorimeter, surface plasmon resonance instrument, or mass spectroscopy instrument.

In other embodiments, the second set of data consists of diffusion rate or solubility measurements obtained, e.g., using column chromatography (e.g., involving a hydrophobic, anion-exchange, cation-exchange, or size exclusion column mounted on, e.g., an HPLC instrument), a diffusion barrier instrument, a solubility instrument, or a capillary electrophoresis instrument. In preferred embodiments, at least some of the diffusion rate or solubility measurements are obtained in parallel, e.g., using a multi-cell or multi-channel instrument, such as a multi-cell or multi-channel column chromatography instrument, diffusion barrier instrument, solubility instrument, or capillary electrophoresis instrument.

In still other embodiments, the second set of data consists of biological (e.g., cell-based or animal-based assay) measurements obtained, e.g., using a visual imaging device (e.g., for counting cells, e.g., stained cells), a spectrophotometer, a spectropolorimeter, a fluorimeter, or a calorimeter. In preferred embodiments, at least some of the biological measurements are obtained in parallel, e.g., using a using a multi-cell or multi-cannel instrument, or an automated device, e.g., an automated imaging device.

In some embodiments, the second set of data, e.g., thermodynamic, spectroscopic, chromatographic, or biological (e.g., from a cell-based or animal-based assay) measurements, includes a single measurement for each compound in the second set of training compounds. In preferred embodiments, the second set of data includes a plurality of measurements, e.g., 2, 3, 4, 5, or more measurements, for each compound in the second set of training compounds.

In some embodiments, the second set of data, e.g., thermodynamic, spectroscopic, chromatographic, or biological (e.g., from a cell-based or animal-based assay) measurements, provides information relevant to therapeutic potency, e.g., binding affinity, of a chemical compound, e.g., a small molecule, protein (e.g., a peptide or modified peptide), or nucleic acid molecule, with respect to an interaction partner, e.g., a molecule (e.g., a protein, lipid, or nucleic acid molecule), a supramolecular structure (e.g., a protein complex, lipid monolayer, lipid bilayer, an in vitro or in vivo membrane system, a protein-nucleic acid complex, or any combination thereof), or a cell. In preferred embodiments, the measurements that provided information about therapeutic potency are thermodynamic measurements, e.g., measurements of ΔH, ΔG, ΔS, equilibrium binding constants, ΔCp, and/or ΔV. In preferred embodiments, the measurements that provide information about therapeutic potency include measurements of ΔH. In particularly preferred embodiments, the measurements that provide information about therapeutic potency include measurements of ΔH, ΔG, and ΔS.

In other embodiments, the second set of data, e.g., thermodynamic, spectroscopic, chromatographic, or biological (e.g., from a cell-based or animal-based assay) measurements, provides information about one or more ADMET properties, e.g., absorption, distribution, metabolism, excretion, or toxicity, of a chemical compound, e.g., a small molecule, protein (e.g., a peptide or modified peptide), or nucleic acid molecule. In preferred embodiments, the ADMET property is absorption, e.g., as measured by permeability (e.g., cellular or membrane permeability), or toxicity, e.g., as measured by chemical conversion of the chemical compound or cellular toxicity in a cell-based or animal-based assay. In other preferred embodiments, the ADMET properties are absorption and distribution or active and passive diffusion, e.g., as measured by logP or permeability through in vitro or in vivo membrane systems.

In some embodiments, the values that provide information about one or more ADMET properties reflect the interaction of a chemical compound, e.g., a small molecule, protein (e.g., a peptide or modified peptide), or nucleic acid molecule, with an interaction partner, e.g., a molecule (e.g., a protein, lipid, or nucleic acid molecule), a supramolecular structure (e.g., a protein complex, lipid monolayer, lipid bilayer, an in vitro or in vivo membrane system, a protein-nucleic acid complex, or any combination thereof), a cell, or an animal. In other embodiments, the values that provide information about one or more ADMET properties reflect the interaction of a chemical compound, e.g., a small molecule, protein (e.g., a peptide or modified peptide), or nucleic acid molecule, with a solvent or a column (e.g., a hydrophobic, anion-exchange, cation-exchange, or size exclusion column or a capillary electrophoresis device).

In some embodiments, a compound of the second training set is a chemical compound, such as a small molecule, e.g., an organic compound, e.g., a fatty acid molecule, a sugar molecule, a steroid molecule, a hormone, a peptide, or any derivative or combination thereof. In other embodiments, a compound of the second training set is a chemical compound extracted from an animal, plant, fungus, or single cell organism, e.g., a bacterium or protist. In preferred embodiments, a compound of the second training set is a chemical compound that has been synthesized in a laboratory, e.g., by combinatorial chemistry or parallel synthesis.

In preferred embodiments, the second training set includes a plurality of training compounds, e.g., 5, 10, 20, 30, 40, 50, 75, 100, 125, 150, 200, or more training compounds.

In some embodiments, the interaction partner is a protein, e.g., a membrane associated protein (e.g., an adhesion receptor, a growth factor signaling receptor, a G-protein coupled receptor, a glycoprotein, or a transporter), a cytoplasmic protein (e.g., an enzyme, such as a carboxylase or transferase or ribosomal protein, a kinase, a phosphatase, an adapter molecule, a GTPase, or an ATPase), or a nuclear protein (e.g., a transcription factor, polymerase, or chromatin associated protein). In other embodiments, the interaction partner is a lipid, e.g., a modified lipid, e.g., phosphatidyl inositol 4, 5-phosphate or a similar lipid involved in signaling pathways. In other embodiments, the interaction partner is a nucleic acid molecule, e.g., DNA or RNA. In other embodiments, the interaction partner is a supramolecular structure, e.g., a multi-subunit protein complex, a protein-DNA or protein-RNA complex, a lipid membrane (e.g., a micelle, a lipid monolayer, or a lipid bilayer), or any combination thereof. In still other embodiments, the interaction partner is a cell, e.g., a mammalian cell, an insect cell, a fungal cell, a bacterium, or a protist.

In some embodiments, the interaction between one or more training compounds of the second set of training compounds and the second interaction partner includes, e.g., the formation of a chemical bond, e.g., a non-covalent bond (e.g., an ionic bond, van der Waals forces, or a combination thereof) or a covalent bond, between the training compound and the second interaction partner. In other embodiments, the interaction between one or more training compounds of the second set of training compounds and the second interaction partner includes, e.g., the breaking of a chemical bond, e.g., a non-covalent bond (e.g., an ionic bond, van der Waals forces, or a combination thereof) or a covalent bond, on either the training compound, the second interaction partner, or both. In other embodiments, the interaction between one or more training compounds of the second set of training compounds and the second interaction partner includes, e.g., the addition or removal of a chemical group, e.g., a phosphate group, on either the training compound, the second interaction partner, or both. In still other embodiments, the interaction between one or more training compounds of the second set of training compounds and the second interaction partner includes, e.g., the oxidation or reduction of a chemical group, e.g., an alcohol, ketone, or carboxylic acid group, on either the training compound, the second interaction partner, or both.

In preferred embodiments, the second set of data, e.g., thermodynamic, spectroscopic, chromatographic, or biological (e.g., from a cell-based or animal-based assay) measurements, is or was experimentally determined, e.g., by a method including the following steps:

providing, for each training compound of the second set of training compounds, at least one reaction mixture which optionally includes the second interaction partner;

inducing a change, e.g., a thermodynamic transition, in each reaction mixture; and

measuring, for each reaction mixture, the value of at least one parameter, e.g., a thermodynamic, spectroscopic, chromatographic, or biological (e.g., from a cell-based or animal-based assay) parameter, describing the interaction between a training compound and the second interaction partner.

In some embodiments, the change includes altering the concentration or activity of a training compound in the reaction mixture, e.g., via the addition of a training compound to each reaction mixture. In other embodiments, the change includes changing the concentration or activity of the second interaction partner, e.g., via the addition of the second interaction partner to each reaction mixture, or by contacting each reaction mixture with the second interaction partner. In other embodiments, the change includes changing the temperature of each reaction mixture.

In preferred embodiments, a plurality of, e.g., at least 5, 10, 20, 50, 100, 200, or more, measurements of a parameter, e.g., a thermodynamic, spectroscopic, chromatographic, or biological (e.g., from a cell-based or animal-based assay) parameter, are determined simultaneously, e.g., by using high throughput screening techniques, e.g., involving multi-cell or multi-channel instruments, e.g., multi-cell or multi-channel calorimeters, spectrophotometers, spectropolorimeters, fluorimeters, NMR detection instruments, mass spectroscopy, column chromatography instruments, diffusion barrier instruments, solubility instruments, capillary based techniques, microarrays or automated visual imaging devices.

In some embodiments, a plurality of, e.g., at least 5, 10, 20, 50, 100, 200, or more, training compounds from the second set of training compounds are determined simultaneously, e.g., in separate cells of a multicell or multi channel instrument. In other embodiments, a plurality of, e.g. at least 5, 10, 20, 50, or more, measurements of a parameter for a single training compound, e.g., under differing conditions, such as the concentration of the training compound or the interaction partner, or the temperature of the reaction mixture, are determined simultaneously.

In some embodiments, the data about the chemical structures and/or physical properties thereof for the second set of training compounds consists of the three dimensional atomic structures of each of the training compounds. In preferred embodiments, the data about the chemical structures and/or physical properties thereof for the second set of training compounds includes the three dimensional atomic structures of each of the training compounds, as well as information about the conformational freedom of the training compounds, e.g., a conformational ensemble profile. In other preferred embodiments, the data about the chemical structures and/or physical properties thereof for the second set of training compounds includes the three dimensional atomic structures of each of the training compounds, as well as information about relevant physical properties of the training compounds, such as hydrophobicity, dipole moment, solubility, electrostatic potential, permeability or, more generally, any property that can be derived from the chemical structure of a molecule. Relevant physical properties will depend upon the structures of the training compounds of the second set of training compounds and the therapeutic property or properties being predicted by the second module of the modular computational model. Such relevant physical properties can be determined as part of the process of constructing the second module of the modular computational model.

In some embodiments, data about the three-dimensional atomic structure and/or physical properties thereof of the interaction partner is included as part of the process of constructing the second module of the modular computational model. In some embodiments, the three-dimensional atomic structure of the interaction partner is well-defined, e.g., when the interaction partner is a protein, nucleic acid molecule, sugar chain, or any combination thereof, and the three-dimensional atomic structure of the interaction partner has been determined, e.g., using crystallography or multi-dimensional NMR. In other embodiments, the three-dimensional atomic structure of the interaction partner is only partially defined, e.g., when the interaction partner is a collection of lipid molecules, e.g., a micelle, a lipid monolayer, a lipid bilayer, or any membrane having characteristics identical to or consistent with a biological membrane. In some embodiments, data about the three-dimensional atomic structure and/or physical properties thereof of the interaction partner is not included as part of the process of constructing the second module of the modular computational model.

In preferred embodiments, the process of constructing the second module of the modular computational model includes techniques commonly used in the construction of quantitative structure-activity relationship (QSAR) models. In particularly preferred embodiments, the process of constructing the second module of the modular computational model includes techniques used in the construction of free energy force field QSAR (FEFF-QSAR) models, three-dimensional QSAR (3D-QSAR) models, four dimensional QSAR (4D-QSAR) models, or membrane interaction QSAR (MI-QSAR) models. In some embodiments, the process of constructing the second module of the modular computational model includes techniques commonly used in the construction of receptor dependent QSAR models, e.g., FEFF-QSAR models, receptor-dependent 4D-QSAR models, or MI-QSAR models. In other embodiments, the process of constructing the second module of the modular computational model includes techniques commonly used in the construction of receptor independent QSAR models, e.g., receptor independent 3D-QSAR models and receptor independent 4D-QSAR models.

In preferred embodiments, the process of constructing the second module of the modular computational model includes the use, e.g., at least once but preferably multiple times, of a partial least squares regression. For example, the partial least squares regression can be used to correlate the values of the second set of data with the data about the chemical structures and/or physical properties thereof of the compounds of the second set of training compounds. In other preferred embodiments, the process of constructing the second module of the modular computational model includes the use, e.g., at least once but preferably multiple times, of a genetic function algorithm (GFA). For example, the GFA can be used to identify features of the chemical structures, e.g., three-dimensional atom structures, and/or physical properties thereof, e.g., conformational freedom, hydrophobicity, dipole moment, solubility, etc., that correlate best with the values of the second set of data. In particularly preferred embodiments, the process of constructing the second module of the modular computational model includes the use, e.g., the alternating use, of both a partial least squares regression and a GFA.

In some embodiments, the second model can be refined, e.g., after being constructed, by the following method:

obtaining a supplemental second set of data, e.g., composed of data similar to the data of the second set of data, e.g., thermodynamic, spectroscopic, chromatographic, or biological (e.g., from a cell-based or animal-based assay), that describes the interaction between each training compound of a supplemental second set of training compounds, e.g., small molecules, proteins (e.g., peptides or modified peptides), or nucleic acid molecules, that are, e.g., structurally or functionally related to the compounds of the second set of training compounds, and the second interaction partner; and

using the second set of data and the supplemental second set of data, along with data about the chemical structures, e.g., three dimensional atomic structures, and/or physical properties thereof, e.g., conformational freedom, hydrophobicity, dipole moment, solubility, electrostatic potential, permeability and, more generally, any property that can be derived from the chemical structure of a molecule, of the second set of training compounds and the supplemental second set of training compounds, and, optionally, using data about the three dimensional structure and/or physical properties thereof of the second interaction partner, to reconstruct the second computational module, e.g., by the same process used to construct the second computational module;

thereby refining the second module of a modular computational model.

In some embodiments, the supplemental second set of training compounds, e.g., small molecules, proteins (e.g., peptides or modified peptides), or nucleic acid molecules, consists of compounds that are structurally or functionally related to the compounds of the second set of training compounds. In other embodiments, the supplemental second set of training compounds, e.g., small molecules, proteins (e.g., peptides or modified peptides), or nucleic acid molecules, consists of at least some compounds that are identical to some of the compounds of the second set of training molecules. For example, the supplemental second set of data could be obtained to either extend the second set of data, to verify some or all of the measurements of the second set of data, or both.

In preferred embodiments, the supplemental second set of data is obtained experimentally using the same experimental techniques used to produce the second set of data. In other embodiments, the supplemental second set of data is obtained experimentally using experimental techniques different from those used to produce the second set of data, e.g., the experimental techniques can be different approaches to measuring the same value, e.g., thermodynamic, spectroscopic, chromatographic, or biological (e.g., from a cell-based or animal-based assay) value. In some embodiments, the supplemental second set of data is obtained from existing information sources, e.g., databases, scientific publications, or internet webpages.

In preferred embodiments, the second module makes predictions about a therapeutic property (or properties), e.g., therapeutic potency (e.g., receptor affinity) or an ADMET (e.g., absorption, distribution, metabolism, excretion and toxicity) property, of chemical compounds that differs from the therapeutic property (or properties) that the first module makes predictions about for the same chemical compounds. For example, the first module could make predictions about the therapeutic potency of chemical compounds, while the second module could make predictions about one or more ADMET properties of chemical compounds. In other embodiments, the second module makes predictions about a therapeutic property (or properties), e.g., therapeutic potency (e.g., receptor affinity) or an ADMET (e.g., absorption, distribution, metabolism, excretion and toxicity) property, of chemical compounds that is the same, or overlaps with, the therapeutic property (or properties) that the first module makes predictions about for the same chemical compounds. For example, the first module could make predictions about the absorption properties (e.g., membrane permeability) of chemical compounds, while the second module could make predictions about the absorption and distribution (e.g., solubility) properties of the same chemical compounds. Alternatively, the first and second modules could both make predictions about the therapeutic potency (e.g. receptor affinity) of chemical compounds, but the predictions could be based on differing parameters, e.g., thermodynamic measurements and spectroscopic measurements, respectively.

Similarly, in preferred embodiments, the second set of data, e.g., thermodynamic, spectroscopic, chromatographic, or biological (e.g., from a cell-based or animal-based assay) measurements, used in the construction of the second module differs from the first set of data, e.g., thermodynamic, spectroscopic, chromatographic, or biological (e.g., from a cell-based or animal-based assay) measurements, used in the production of the first module. For example, the first set of data could be thermodynamic or spectroscopic data that relates to the therapeutic potency (e.g., binding affinity) of the training compounds of the first set of training compounds with respect to the first interaction partner, while the second set of data could be thermodynamic, spectroscopic or biological data that relates to an ADMET property of the training molecules of the second set of training.

In some embodiments, the first set of training compounds differs, e.g., by one or more training compounds, from the second set of training compounds. In some embodiments, the first set of training compounds completely differs from the second set of training compounds. In still other embodiments, the first set of training molecules is identical to the second set of training molecules.

In some embodiments, the first interaction partner is similar or identical to the second interaction partner, e.g., the first and second interaction partners can be the same protein or complex thereof, or can be, e.g., micelles, lipid bilayers, or cells. In other embodiments, the first interaction partner differs from the second interaction partner. For example, the first interaction partner can be a protein, while the second interaction partner is a lipid bilayer, a cell, or a solvent.

In preferred embodiments, at least one module of a modular computational model predicts the therapeutic potency, e.g., receptor affinity, of chemical compounds. In other preferred embodiments, a modular computational model includes at least two modules, wherein at least one module predicts the therapeutic potency, e.g., receptor affinity, of chemical compounds, and wherein at least one module predicts one or more ADMET properties, e.g., absorption, distribution, metabolism, excretion, and toxicity, of chemical compounds.

In preferred embodiments, for each nth module, wherein n represents the third, fourth, fifth, sixth, etc. module of a modular computational model, the nth module is constructed by a process similar to the process used to construct the second module.

In another aspect, a modular computational model, e.g., a modular computational model constructed as described above, is used to produce one or more structural models, e.g., three-dimensional atomic structure models, that illustrate the relationship between the chemical groups, e.g., hydrogen bond acceptor, hydrogen bond donor, polar, hydrophobic, or charged groups, of a compound's structure and their relationship to one or more of the known or predicted therapeutic properties, e.g., therapeutic potency or an ADMET property, of the compound. For example, groups that are particularly important with respect to therapeutic potency, e.g., receptor affinity, could be highlighted, or groups that are particularly disruptive with respect to therapeutic potency could be highlighted, or both types of groups could be highlighted. Alternatively, groups that are particularly important with respect to one therapeutic property, e.g., therapeutic potency (e.g., receptor affinity), and a second therapeutic property, e.g., an ADMET property, could be highlighted. In some embodiments, the structural models depict compounds that are members of the first set of training compounds. In other embodiments, the structural models depict compounds that are members of, e.g., the second, third, fourth, fifth, sixth, etc., set of training compounds. In other embodiments, the structural models depict one or more compounds that are not members of any of the sets of training compounds used to construct the modules of the modular computational model, but instead have a generic structure common to at least some of the compounds of one or more sets of training compounds.

In another aspect, the invention features methods of evaluating a plurality of test structures, e.g., chemical compounds, e.g., small molecules, proteins (e.g., peptides or modified peptides), or nucleic acid molecules, for one or more therapeutic properties, e.g., therapeutic potency (e.g., receptor affinity) or an ADMET property (e.g., absorption, distribution, metabolism, excretion, and toxicity), using one or more modular computational models. The methods include:

a) providing a first modular computational model, which can be constructed, e.g., by any of the methods described above;

b) providing the chemical structure, e.g., three dimensional atomic structure, and/or physical properties thereof, e.g., conformational freedom, hydrophobicity, dipole moment, solubility, electrostatic potential, permeability and, more generally, any property that can be derived from the chemical structure of a molecule, for all or a part of each member of the plurality of test structures;

c) applying the first modular computational model to each member of the plurality of test structures, e.g., to the chemical structures and/or physical properties thereof of all or a part of each member of the plurality of test structures, to obtain a first set of predicted values, e.g., thermodynamic, spectroscopic, chromatographic, or biological (e.g., from a cell-based or animal-based assay) values, describing the interaction between each member of the plurality of test structures and one or more interaction partners; and optionally analyzing the values, e.g., by:

d) comparing the predicted values, e.g., thermodynamic, spectroscopic, chromatographic, or biological (e.g., from a cell-based or animal-based assay) values, from the first set of predicted values with one or more reference values; or

e) ranking the predicted values, e.g., thermodynamic, spectroscopic, chromatographic, or biological (e.g., from a cell-based or animal-based assay) values, from the first set of predicted values,

thereby evaluating one or more therapeutic properties of the plurality of test structures.

In preferred embodiments, the first modular computational model is constructed as part of the methods of the invention. In other embodiments, the first modular computational model already exists and is merely provided as part of the methods of the invention. In particularly preferred embodiments, the first modular computational model is constructed as described above.

In some embodiments, the first modular computational model consists of a single module. In other embodiments, the first modular computational model consists of two or more modules. In preferred embodiments, at least one module of the first modular computational model predicts the therapeutic potency, e.g., receptor affinity, of chemical compounds. In other preferred embodiments, the first modular computational model includes at least two modules, wherein at least one module predicts the therapeutic potency, e.g., receptor affinity, of chemical compounds. In other preferred embodiments, the first modular computational model includes at least two modules, wherein at least one module predicts the therapeutic potency, e.g., receptor affinity, of chemical compounds, and wherein at least one module predicts one or more ADMET properties, e.g., absorption, distribution, metabolism, excretion, and toxicity, of chemical compounds. In still other preferred embodiments, the first modular computational model includes more than two modules, wherein at least one module predicts the therapeutic potency, e.g., receptor affinity, of chemical compounds, and wherein at least one module predicts one or more ADMET properties, e.g., absorption, distribution, metabolism, excretion, and toxicity, of chemical compounds.

In some embodiments, the first set of predicted values includes a single predicted value for each test structure of the plurality of test structures. In other embodiments, the first set of predicted values includes two or more predicted values for each test structure of the plurality of test structures. In general, the number of predicted values in the first set of predicted values that relate to each test structure of the plurality of test structures is greater than or equal to the number of modules that constitute the first modular computational model.

In preferred embodiments, the first set of predicted values provides an indication of the therapeutic potency, e.g., receptor affinity, of each test structure in the plurality of test structures. In other preferred embodiments, the first set of predicted values provides an indication of the therapeutic potency, e.g., receptor affinity, and at least one other therapeutic property, e.g., an ADMET property, e.g., absorption, distribution, metabolism, excretion, and toxicity, of each test structure in the plurality of test structures. In other preferred embodiments, the first set of predicted values provides an indication of the therapeutic potency and one or more ADMET properties of each test structure in the plurality of test structures. In still other preferred embodiments, the first set of predicted values provides an indication of the therapeutic potency and at least two ADMET properties of each test structure in the plurality of test structures.

In some embodiments, some or all of the predicted values, e.g., thermodynamic, spectroscopic, chromatographic, or biological (e.g., from a cell-based or animal-based assay) values, of the first set of predicted values are compared with a reference value. In general the number of reference values will match the number of modules in the modular computational model, and predicted values originating from a specific module will only be compared with the appropriate reference value. In some embodiments, compounds that have a predicted value that is above the relevant reference value with be scored as having a desirable property, e.g., a desirable therapeutic potency or a desirable ADMET property. In other embodiments, compounds that have a predicted value that is below the relevant reference value will be scored as having a desirable property, e.g., a desirable therapeutic potency or a desirable ADMET property.

In some embodiments, some or all of the predicted values, e.g., thermodynamic, spectroscopic, chromatographic, or biological (e.g., from a cell-based or animal-based assay) values, of the first set of predicted values will be ranked relative to one another. In general, predicted values will only be ranked relative to other predicted values that were generated by the same module of the modular computational model. Thus, in some embodiments, there will be at least as many rankings of the predicted values as there are modules in the modular computational model. In some embodiments, only the predicted values originating from certain modules, e.g., modules that predict pharmaceutical potency, will be ranked relative to one another. In some embodiments, compounds that have a predicted value that is ranked within the top, e.g., 1%, 5%, 10%, 20%, 30%, 40%, or 50%, of predicted values will be scored as having a desirable property, e.g., a desirable therapeutic potency or a desirable ADMET property. In other embodiments, compounds that have a predicted value that is ranked within the bottom, e.g., 1%, 5%, 10%, 20%, 30%, 40%, or 50%, of predicted values will be scored as having a desirable property, e.g., a desirable therapeutic potency or a desirable ADMET property.

In some embodiments, the methods of evaluating a plurality of test structures, e.g., chemical compounds, e.g., small molecules, proteins (e.g., peptides or modified peptides), or nucleic acid molecules, for one or more therapeutic properties, e.g., therapeutic potency (e.g., receptor affinity) or an ADMET property (e.g., absorption, distribution, metabolism, excretion, and toxicity), further include using a second modular computational model. The methods include:

a) providing a second modular computational model, which can be constructed, e.g., by any of the methods described above;

b) providing the chemical structure, e.g., three dimensional atomic structure, and/or physical properties thereof, e.g., conformational freedom, hydrophobicity, dipole moment, solubility, electrostatic potential, permeability and, more generally, any property that can be derived from the chemical structure of a molecule, for all or a part of each member of the plurality of test structures;

c) applying the second modular computational model to each member of the plurality of test structures, e.g., to the chemical structures and/or physical properties thereof of all or a part of each member of the plurality of test structures, to obtain a second set of predicted values, e.g., thermodynamic, spectroscopic, chromatographic, or biological (e.g., from a cell-based or animal-based assay) values, describing the interaction between each member of the plurality of test structures and one or more interaction partners; and optionally analyzing the values, e.g., by:

d) comparing the predicted values, e.g., thermodynamic, spectroscopic, chromatographic, or biological (e.g., from a cell-based or animal-based assay) values, from the second set of predicted values with one or more reference values; or

e) ranking the predicted values, e.g., thermodynamic, spectroscopic, chromatographic, or biological (e.g., from a cell-based or animal-based assay) values, from the second set of predicted values,

thereby evaluating at least two therapeutic properties of the plurality of test structures.

In preferred embodiments, the second modular computational model is constructed as part of the methods of the invention. In other embodiments, the second modular computational model already exists and is merely provided as part of the methods of the invention. In particularly preferred embodiments, the second modular computational model is constructed as described above.

In some embodiments, the second modular computational model consists of a single module. In other embodiments, the second modular computational model consists of two or more modules. In preferred embodiments, at least one module of the second modular computational model predicts one or more ADMET properties, e.g., absorption, distribution, metabolism, excretion, and toxicity, of chemical compounds. In other preferred embodiments, the second modular computational model includes a t least two modules, wherein at least one module predicts one or more ADMET properties, e.g., absorption, distribution, metabolism, excretion, and toxicity, of chemical compounds. In other preferred embodiments, the second modular computational model includes two or more modules, wherein at least two of the modules predict one or more ADMET properties, e.g., absorption, distribution, metabolism, excretion, and toxicity, of chemical compounds. In other embodiments, the second modular computational model includes a module that predicts the therapeutic potency, e.g., receptor affinity, of chemical compounds. In other embodiments, the second modular computational model includes at least two modules, wherein at least one module predicts the therapeutic potency, e.g., receptor affinity, of chemical compounds, and wherein at least one module predicts one or more ADMET properties, e.g., absorption, distribution, metabolism, excretion, and toxicity, of chemical compounds. In still other embodiments, the second modular computational model includes more than two modules, wherein at least one module predicts the therapeutic potency, e.g., receptor affinity, of chemical compounds, and wherein at least one module predicts one or more ADMET properties, e.g., absorption, distribution, metabolism, excretion, and toxicity, of chemical compounds.

In some embodiments, the second set of predicted values includes a single predicted value for each test structure of the plurality of test structures. In other embodiments, the second set of predicted values includes two or more predicted values for each test structure of the plurality of test structures. In general, the number of predicted values in the second set of predicted values that relate to each test structure of the plurality of test structures is greater than or equal to the number of modules that constitute the second modular computational model.

In preferred embodiments, the second set of predicted values provides information about one or more ADMET properties, e.g., absorption, distribution, metabolism, excretion, and toxicity, of each test structure in the plurality of test structures. In other preferred embodiments, the second set of predicted values provides an indication of the therapeutic potency, e.g., receptor affinity, and information about one or more ADMET properties, e.g., absorption, distribution, metabolism, excretion, and toxicity, of each test structure in the plurality of test structures. In other preferred embodiments, the second set of predicted values provides an indication of the therapeutic potency and information about at least two ADMET properties of each test structure in the plurality of test structures. In other embodiments, the second set of predicted values provides an indication of the therapeutic potency, e.g., receptor affinity, or each test structure in the plurality of test structures.

In some embodiments, some or all of the predicted values, e.g., thermodynamic, spectroscopic, chromatographic, or biological (e.g., from a cell-based or animal-based assay) values, of the second set of predicted values are compared with a reference value. In general the number of reference values will match the number of modules in the second modular computational model, and predicted values originating from a specific module will only be compared with the appropriate reference value. In some embodiments, compounds that have a predicted value that is above the relevant reference value with be scored as having a desirable property, e.g., a desirable therapeutic potency or a desirable ADMET property. In other embodiments, compounds that have a predicted value that is below the relevant reference value will be scored as having a desirable property, e.g., a desirable therapeutic potency or a desirable ADMET property.

In other embodiments, some or all of the predicted values, e.g., thermodynamic, spectroscopic, chromatographic, or biological (e.g., from a cell-based or animal-based assay) values, of the second set of predicted values will be ranked relative to one another. In general, predicted values will only be ranked relative to other predicted values that were generated by the same module of the second modular computational model. Thus, in some embodiments, there will be at least as many rankings of the predicted values as there are modules in the second modular computational model. In some embodiments, only the predicted values originating from certain modules, e.g., modules that predict an ADMET property, will be ranked relative to one another. In some embodiments, compounds that have a predicted value that is ranked within the top, e.g., 1%, 5%, 10%, 20%, 30%, 40%, or 50%, of predicted values will be scored as having a desirable property, e.g., a desirable therapeutic potency or a desirable ADMET property. In other embodiments, compounds that have a predicted value that is ranked within the bottom, e.g., 1%, 5%, 10%, 20%, 30%, 40%, or 50%, of predicted values will be scored as having a desirable property, e.g., a desirable therapeutic potency or a desirable ADMET property.

In preferred embodiments, the second modular computational model includes one or more modules that predict the values of one or more therapeutic properties, e.g., therapeutic potency (e.g., receptor affinity) or an ADMET property (e.g., absorption, distribution, metabolism, excretion, and toxicity), wherein at least one of the modules of the second modular computational model is distinct from the modules of the first modular computational model. For example, the first modular computational model can include at least one module that predicts the therapeutic potency of each test structure of the plurality of test structures, while the second modular computational model can include at least one module that predicts one or more ADMET properties of each test structure of the plurality of test structures, or vice versa.

In some embodiments, the methods of evaluating a plurality of test structures, e.g., chemical compounds, e.g., small molecules, proteins (e.g., peptides or modified peptides), or nucleic acid molecules, for one or more therapeutic properties, e.g., therapeutic potency (e.g., receptor affinity) or an ADMET property (e.g., absorption, distribution, metabolism, excretion, and toxicity), further include providing and applying, e.g., a third, fourth, fifth, sixth, etc., modular computational model. In preferred embodiments, each additional modular computational model after the second is provided, applied, and optionally evaluated in the same manner as the second modular computational model. In preferred embodiments, each additional computational model after the second includes a module, e.g., that predicts a therapeutic property, e.g., therapeutic potency or an ADMET property, that is not present in any of the earlier modules, and thus provides a new set of predicted values.

In some embodiments, a compound described by the plurality of test structures is a chemical compound such as a small molecule, e.g., an organic compound, e.g., a fatty acid molecule, a sugar molecule, a steroid molecule, a hormone, a peptide, or any derivative or combination thereof. In other embodiments, a compound described by the plurality of test structures is a chemical compound extracted from an animal, plant, fungus, or single cell organism, e.g., a bacterium or protist. In preferred embodiments, a compound described by the plurality of test structures is a chemical compound that has been synthesized in a laboratory, e.g., by combinatorial chemistry or parallel synthesis. In other preferred embodiments, a compound described by the plurality of test structures is a virtual compound. In still other preferred embodiments, a compound described by the plurality of test structures is a chemical compound that is structurally related (e.g., similar in three dimensional atomic structure or similar in general structure (e.g., amphipathic)) to one or more molecules in one of the first, second, third, fourth, etc. sets of training structures used to construct the modules of the modular computational model.

In preferred embodiments, providing the chemical structure for all or part of each member of the plurality of test structures involves providing a data structure, e.g., a database, e.g., a computer database, that describes the chemical structure, e.g., three-dimensional atomic structure, and/or physical properties thereof, e.g., conformational freedom, hydrophobicity, dipole moment, solubility, etc., for all or part of each member of the plurality of test structures. In some embodiments, the data structure describing the chemical structure and/or physical properties thereof for all or part of each member of the plurality of test structures is constructed as part of the methods of evaluating the plurality of test structures. For example, the data structure can be generated by collecting information, e.g., structural information and/or related physical properties, about many different chemical compounds known in the art, it can be generated by making up new chemical structures (e.g., virtual compounds), e.g., on a computer, or it can be generated by both of these approaches. In other embodiments, the data structure already exists and is merely obtained and then provided as part of the methods of evaluating the plurality of test structures. In still other embodiments, the data structure exists in part and is added to, e.g., by gathering information about additional chemical compounds, making up new chemical structures (e.g., virtual compounds), or manipulating the existing database (e.g., providing information about the physical properties, e.g., conformational freedom, hydrophobicity, dipole moment, solubility, etc., of the chemical compounds.

In preferred embodiments, the plurality of test structures includes at least 100, 200, 300, 400, 500, 1,000, 2,000, 5,000, 10⁴, 10⁵, 10⁶, 10⁷, 10⁸, 10⁹, or more different chemical structures that represent real or virtual chemical compounds.

In some embodiments, a subset of the plurality of test structures is identified that includes all of the test structures that are predicted to have at least one desirable property, e.g., a desirable therapeutic potency or a desirable ADMET property, as predicted by any module of any modular computational model applied to the plurality of test structures. In preferred embodiments, a subset of the plurality of test structures is identified that includes all of the test structures that are predicted to have at least two desirable properties, as predicted by any pair of modules included as part of the modular computational models applied to the plurality of test structures. In particularly preferred embodiments, a subset of the plurality of test structures is identified that includes all of the test structures that are predicted to have a desirable therapeutic potency and at least one desirable ADMET property. In other particularly preferred embodiments, a subset of the plurality of test structures is identified that includes all of the test structures that are predicted to have a desirable therapeutic potency and two or more desirable ADMET properties.

In some embodiments, the methods of evaluating a plurality of test structures further include using the predicted values to produce one or more structural models, e.g., three-dimensional atomic structure models, that illustrate the relationship between the chemical groups, e.g., hydrogen bond acceptor, hydrogen bond donor, polar, hydrophobic, or charged groups, of a compound's structure and their relationship to one or more of the known or predicted therapeutic properties, e.g., therapeutic potency or an ADMET property, of the compound. For example, groups that are particularly important with respect to therapeutic potency, e.g., receptor affinity, could be highlighted, or groups that are particularly disruptive with respect to therapeutic potency could be highlighted, or both types of groups could be highlighted. Alternatively, groups that are particularly important with respect to one therapeutic property, e.g., therapeutic potency (e.g., receptor affinity), and a second therapeutic property, e.g., an ADMET property, could be highlighted. In some embodiments, the structural models depict compounds that are members of the plurality of test structures. In preferred embodiments, the structural models depict compounds that are members of the plurality of test structures predicted to have at least one desirable therapeutic property, e.g., therapeutic potency or an ADMET property. In other embodiments, the structural models depict one or more compounds that are not members of the plurality of test structures, but instead have a generic structure common to many members of the plurality of test structures.

In some embodiments, the methods of evaluating a plurality of test structures further include producing a data structure, e.g., a database, e.g., a computer-based database, that stores the predicted values from at least one module of one modular computational model used in the evaluation of each structure of the plurality of test structures. In preferred embodiments, the data structure includes the predicted values of all of the modules of the modular computational models used in the evaluation of each structure of the plurality of test structures. In other embodiments, the methods of evaluating a plurality of test structures further include producing a data structure, e.g., a database, e.g., a computer-based database, that stores the predicted values from at least one module of one modular computational model used in the evaluation of a subset of structures of the plurality of test structures, e.g., a subset of structures predicted to have one or more desirable therapeutic properties. In some embodiments, the data structure includes additional information about the predicted values associated with each structure in the database, e.g., information about the relative ranking of the predicted values or a comparison of the values to a reference value.

In a preferred embodiment, the methods further include selecting, e.g., from a library of structures, a candidate structure, e.g., a structure predicted to have one or more desirable therapeutic properties, and further evaluating the selected candidate structure, e.g., by retesting, confirming, or testing anew, for a therapeutic property, which can be the predicted desirable therapeutic property or some other property, in an in vitro or in vivo, e.g., cell- or animal based, system.

As used herein, a “dersirable therapeutic property” is a therapeutic property that would tend to improve the efficacy of a drug candidate. For example, desirable therapeutic potency refers high ligand-receptor affinity. Similarly, desirable ADMET properties are those properties which allow a drug to remain in the circulation, target the intended receptor, and not cause any adverse side effects, such a an immune reaction or cellular toxicity.

As used herein, a “high throughput instrument” is any instrument that can be used to measure, either directly or indirectly, a pharmaceutical property of a drug, wherein the instrument is capable of performing a plurality, e.g., at least 5, 10, 15, 20, 25, or more, of measurements simultaneously or, alternatively, is capable of automatically performing a plurality, e.g., 5, 10, 20, 50, 100, 1000, or more, of measurements in a sequential manner and with little or no supervision while the measurements are being performed.

As used herein, the term “virtual compound” refers to any chemical compound, whether the compound exists in nature or not, that may be structurally represented, e.g., in a database, e.g., a computer database.

As used herein, the term “thermodynamic transition” refers to any change in a reaction mixture, e.g., the addition or removal of heat, the addition of a training compound, the addition of an interaction partner, or the addition of some other compound (e.g., a salt, acid, or base), that is capable of producing a measurable thermodynamic change in the reaction mixture.

As used herein, the term “scoring function” refers to an algebraic equation that attempts to relate a property of a chemical compound, e.g., a training compound, to the structure, e.g., three-dimensional atomic structure, and/or physical properties thereof, of the chemical compound.

As used herein, the phrase “value of a therapeutic property” refers to measurement, e.g., a thermodynamic, spectroscopic, chromatographic, or biological (e.g., from a cell-based or animal-based assay) measurement, with respect to a chemical compound that can be related, either directly or through mathematical manipulation, to a therapeutic property, e.g., therapeutic potency (e.g., receptor affinity) or an ADMET property (e.g., absorption, distribution, metabolism, excretion and toxicity), of the chemical compound.

The methods of the present invention offer a number of advantages with respect to rapidly identifying high quality drug candidates. The methods include, for example, the generation of experimental data and/or can incorporation of experimental data obtained from many different sources. The experimental data can be of many different types. For example, the experimental data can be measurements of the binding of a plurality of chemical compounds to an interaction partner, such as a therapeutic protein target or a macromolecular structure, e.g., a protein complex, a nucleic acid molecule, a micelle, a lipid bilayer, or combinations thereof. Alternatively, the experimental data can be measurements relating to the ADMET properties of a set of molecules, such as membrane permeability, solvent solubility, or toxicity. The experimental data, whether gathered, e.g., from scientific publications, generated explicitly for the methods of the invention, or both, can subsequently be processed using computational algorithms to develop modular computational models, or scoring functions, for the prediction of data of the same type for molecules that have not been experimentally assayed. The prediction methods can be applied to many different molecules, including molecules that are readily available, as well as virtual molecules. The experimental and computational methods of the invention can be applied as high throughput screens to identify drug candidates in pharmaceutical applications.

A primary, but not a restrictive, application of the process is to perform high throughput screens (HTSs) of molecules, e.g., ligands, for their ability to bind to interaction partners, e.g., protein or macromolecular receptors, e.g., individual proteins, protein complexes, nucleic acid molecules, micelles, lipid bilayers, or combinations thereof, as part of a new drug discovery process. See A. J. Hopfinger and J. S. Duca, Curr. Opin. Biotech., 11:97-103 (2000), the contents of which are incorporated herein by reference. Combinatorial chemistry and/or parallel synthesis technologies applied to lead optimization in new drug discovery can also employ the methods of the invention. See W. F. Zheng, S. J. Cho, A. Trophsa, J.Chem. Inf. Comput. Sci., 38: 251-258 (1998), the contents of which are incorporated herein by reference. Experimental binding measurements of, for example, a set of ligands with a receptor, can be used to rank and sort the ligands in terms of their binding potency to a given receptor. Such binding measurements can also be used to calibrate computational scoring functions to accurately and reliably predict the binding measures of ligands that have not been experimentally analyzed, including virtual ligands. See W. P. Walters, M. T. Stahl, M. A. Murko, Drug Discovery Today, 3:160-194 (1998), and A. J. Hopfinger, A. Reaka, P. Venkatarangan, J. S. Duca, S. Wang, J. Chem. Inf. Comput. Sci. 39: 1151-1160 (1999), the contents of which are incorporated herein by reference. Thus, the methods of the present invention can be used as adjuncts to, as well as replacements for, current assays and screens used in both HTS and combinatorial chemistry methods prevalent in the pharmaceutical and biotechnology industries. In addition, the methods of the invention can include, for example, using the calibrated and optimized scoring functions for computational screening of molecules, e.g., from libraries of molecules, including virtual molecules, to define subsets of molecules that can subsequently be assayed experimentally. Such subsequently obtained experimental data can be used to validate and refine the computational models in a recursive manner.

Scoring functions based upon algorithms from both structure-based design methods and quantitative structure-activity relationship (QSAR) analyses can be calibrated using the experimental binding data that has been either generated as part of, or gathered for, the methods of the invention.

The methods of the invention uniquely incorporate, but are not restricted to, the experimental determination of thermodynamic binding measurements, such as ΔG, ΔS, ΔH, equilibrium constants, between molecules (e.g., ligands) and potential interaction partners, such as protein or macromolecular receptors, e.g., individual proteins, protein complexes, nucleic acid molecules, micelles, or lipid bilayers. Thermodynamic binding measurements determined, e.g., for ligand-receptor binding, can replace, or serve as an adjunct to, the screens and assays employed in HTS and combinatorial chemistry experiments. Similarly, thermodynamic binding measures determined, e.g., for membrane permeability or solvent solubility, can replace, or serve as an adjunct to, the screens and assays used for determining the ADMET properties of a drug candidate.

Thermodynamic binding data generated by calorimetric screening is much richer in the information needed to identify drug candidates than the data generated in current in vitro biological screens, including those screens typically used in HTS and combinatorial chemistry applications. Calorimetric measurements include, e.g., determination of the overall free energy (ΔG), enthalpy (ΔH), and entropy (ΔS) of the ligand-receptor binding process, as well as their respective temperature dependencies. Moreover, these same thermodynamic quantities can be determined for the component interactions of the overall ligand-receptor binding process by extended applications of this multiplex process. The component interactions include direct ligand-receptor binding, ligand and receptor desolvation, change in ligand conformation upon binding and change in receptor geometry upon binding. The free energy, enthalpy and entropy of ligand-receptor binding provides unique data to identify the best ligands, or “hits”, from a library to use in defining molecular structure requirements—the pharmacophore—for drug-candidate compounds.

Construction of the modular computational models can include the scaling and calibration of force fields, by applying experimental thermodynamic and spectroscopic data, for the accurate computational prediction of the binding interactions of interacting chemical systems, such as ligand-receptor binding. The geometry of the receptor used in the force field calibrations will normally come from X-ray, NMR, homology model building and/or sequence-structure predictions. However, any other means of obtaining receptor geometry can be accommodated by the process.

Scaled force fields can be applied in the virtual high throughput screening (VHTS) of actual or virtual compound libraries. This form of VHTS may applied as a preprocessing screen to actual compound synthesis and screening, or a substitute for experimental HTS.

In combination with the screening of compounds for therapeutic potency (e.g., high affinity ligand-receptor binding), the methods incorporated high throughput thermodynamic and spectroscopic screening of the ADMET (absorption, distribution, metabolism, excretion and toxicological) properties of drug-candidate molecules. Such drug-candidate molecules can include, but not are not limited to, ligands found to bind tightly to a receptor using the high throughput thermodynamic and spectroscopic screening of the binding interaction between two molecular entities or predicted to bind tightly to a receptor using the described modular computational models.

It is recognized that multiplex, high throughput instruments can increase the number of compounds screened, e.g., for thermodynamic or spectroscopic binding data, or membrane permeability, solvent solubility, or toxicity data, in a manner directly proportional to the number of data channels on the instrument. The result is a reduction in the time that is required to experimentally screen molecules, develop and refine related computational models, and screen sets of test molecules, which has the benefit of reducing costs in the pharmaceutical industry. In addition, by increasing the number of compounds screened for thermodynamic or spectroscopic binding data, high throughput instruments can bring about improvements in the accuracy of the scoring functions that constitute the modules of the modular computational models.

In particular, multichannel parallel calorimeters can be used to determine the thermodynamic binding properties of, e.g., a set of molecules, such as a training set of molecules, and a common interaction partner, e.g., a therapeutic protein target or a macromolecular structure, e.g., a protein complex, a nucleic acid molecule, a micelle, a lipid bilayer, or combinations thereof. The high throughput screening capabilities of multiplex calorimetric devises can be used to determine either single-point thermodynamic measurements of large numbers of distinct interacting chemical systems in short times, or many-point thermodynamic measurements of a single interacting chemical system in a short time.

Thus, the methods of the present invention can include one or more of the following steps:

1. The determination of thermodynamic, spectroscopic, and other property measurements, e.g., therapeutic property measurements, for one or more sets of molecules, e.g., test sets of molecules, using instruments constructed to perform the measurements in highly parallel, multiplex processing modes. In some cases, this step can be supplemented with, or even supplanted by, property measurements obtained, e.g., from scientific publications, for a set of molecules.

2. The use of experimental property measurements, e.g., thermodynamic (e.g., free energy, enthalpy and entropy of binding) and spectroscopic measurements, or measurements of membrane permeability, solvent solubility, or toxicity, to generate modular computational models (one or more scoring functions) that predict such properties for molecules that have not been experimentally evaluated.

3. The use of modular computational models to reliably and robustly conduct virtual high throughput screens (VHTSs) on one or more sets of molecules, e.g., test sets or libraries of molecules, and thereby evaluate the properties of the test molecules and identify those test molecules which may have desirable properties.

4. The use of the methods of step 1 to experimentally evaluate test molecules that are predicted to have desirable properties, e.g., molecules identified as having desirable properties in step 3.

5. The use of the experimental property measurements determined in step 4 to refine the model of step 2.

6. The use of any of steps 2-5 in conjunction with traditional high throughput screens.

7. The use of modular computational models having two or more modules, or the combined use of two or more modular computational models having at least one module each, according to steps 2-5, to predict, e.g., thermodynamic and spectroscopic estimates of both therapeutic potency (e.g., ligand-receptor binding interactions) and one or more ADMET properties, and thereby perform overall lead optimization on one or more sets of test molecules.

The details of one or more embodiments of the invention are set forth in the accompanying drawings and the description below. Other features, objects, and advantages of the invention will be apparent from the description and drawings, and from the claims.

### DETAILED DESCRIPTION

### Pharmaceutical Properties of Chemical Compounds

The important pharmaceutical properties of drug candidates include, but are not restricted to, pharmaceutical potency and ADMET properties. As used herein, “pharmaceutical potency” refers to the affinity, or binding energy, associated with the interaction between two compounds, e.g., a chemical compound, such as a ligand, and a potential target, e.g., a receptor. The affinity of a drug candidate for its intended target is a major determinant of how successful the drug candidate will be when administered to a patient. In general, drug candidates that bind to their intended target with high affinity can be administered at lower doses, thereby reducing the risk of side effects while maximizing the chance that the drug candidate will bind specifically to its intended target.

Successful drug-candidate ligands should not only bind with high affinity to their therapeutic target, but should also possess essential ADMET properties (Absorption, Distribution, Metabolism, Excretion, and Toxicity). Proper ADMET properties control the optimal expression of therapeutic potency and minimize side effects of the drug, e.g., ligand. Absorption refers to processes whereby the drug candidate binds non-specifically to molecules in the body, e.g., proteins membranes, etc. The absorption properties of a compound can impact its efficacy, as a compound that is readily absorbed by the body may not be able to reach its intended target. Alternatively, a compound may need to be absorbed by cells so as to reach an intracellular target, e.g., if the compound is a steroid or steroid derivative. Distribution, which is related absorption, refers to where a drug candidate accumulates in the body of a patient, e.g., widespread distribution, accumulates in the liver, accumulates in the kidney, does or does not cross the blood brain barrier. If a compound is not able to reach the tissue that contains its target, then the compound will not be an effective drug. Metabolism refers to the body's ability to degrade a drug candidate. If a drug candidate is readily metabolized, it may not have time to reach its intended target before losing some or all of its activity. Furthermore, a drug candidate can be metabolized into a derivative compound that is toxic to the body. Excretion refers to how quickly a drug candidate is removed from the body. Compounds that have a short half-life typically need to be administered more often and at higher doses to ensure that some of the compound reaches its target. Finally, toxicity refers to side effects associated with administering a drug candidate to a patient. Foreign compounds can disrupt many different aspect of cellular behavior, giving rise to cell death (e.g., chemotherapeutic drugs) or stimulating an immune response, which can aggravate a patient's illness.

Clearly, to identify drug candidates that have the most promise, it is necessary to consider many different pharmaceutical properties during the screening process.

### Measuring Pharmaceutical Properties

Many different assays have been developed that measure, either directly or indirectly, some aspect of a drug candidate's pharmaceutical properties. Any assay that can provide a measurement of one or more pharmaceutical properties of a drug candidate can be used to generated data that is suitable for use in the methods of the invention. Specific examples are described below. The measurements that are used to describe the pharmaceutical properties of compounds include, but are not limited to, thermodynamic, spectroscopic, chromatographic, and biological (e.g., from a cell-based or animal-based assay) measurements

### Therapeutic Potency

Thermodynamic measurements provide information about how molecules interact with one another. Thus, thermodynamic measurements can be used to describe or measure, in whole or in part, many different properties of a drug candidate, including therapeutic potency, absorption, distribution, and toxicity. Thermodynamic measurements include, but are not limited to, measurements of free energy (ΔG), enthalpy (ΔH), entropy (ΔS), binding constants, heat capacity (ΔCp), and volume (ΔV).

Thermodynamic measurements, especially measurements of free energy, enthalpy, entropy, and binding constants, have been used extensively to describe the interactions of two molecule systems, such as that of a ligand and receptor. The change in enthalpy (ΔH) is a particularly useful thermodynamic measurement when considering ligand-receptor interactions, as it is a direct measurement of binding specificity. Similarly the change in free energy (ΔG) is a useful thermodynamic measurement, as it provides a measure of binding affinity. Thus, thermodynamic measurements such as ΔH, ΔG, and ΔS, and especially the combination of the three, can be used to measure the pharmaceutical potency of a drug candidate. Measurement of thermodynamic parameters such as ΔH, ΔG, and ΔS can be performed using many different instrument, particularly calorimeters, e.g., differential scanning calorimeters or isothermal titration calorimeters, but also spectroscopic instruments, e.g., spectrophotometers, spectropolorimeters, fluorimeters, or NMR detection instruments.

The advent of highly parallel, multichannel instrumentation for obtaining thermodynamic parameters of binding interactions between molecular and/or chemical entities has the potential to enable more efficient, effective high throughput screening processes and thereby extremely expedite the process of drug design, development and discovery. Among the most promising of these instruments currently being contemplated or already developed are the multi-cell differential scanning calorimeter (MC-DSC) and multi-cell isothermal titration calorimeter (MC-ITC). These instruments will be capable of multiplex (multiple scans simultaneously) measurements of thermodynamic parameters of biological macromolecules and their complexes with other macromolecules, small molecules, ligands and drugs.

In an MC-DSC instrument, the sample temperature of each well is increased identically while the excess heat capacity is monitored as a function of temperature. The temperature dependence of the heat capacity versus temperature is obtained and can be readily dissected, by methods known in the art, to provide the binding constant and corresponding thermodynamic parameters. This instrument can also provide a measure of the difference in heat capacity between the initial and final states, ΔCp which can be equated to the difference in solvent exposed surface area between the bound and unbound states. Thus, indirect structural information can also be obtained.

An MC-ITC instrument determines directly the heat of each reaction between the binding entity and substrate in each sample chamber, at a constant temperature. The binding entitiy is added (titrated) with the substrate (or vice versa) and the heat of the resulting reactions is measured. The measured heat is directly related to the enthalpy of the binding reaction. By conducting ITC measurements at different temperatures, the temperature dependence of the transition enthalpy and entropy can be obtained, which again provides a measure of the ΔCp.

Spectroscopic measurements of absorbance (erg., ultraviolet, visible, infrared light absorbance), emissions (e.g., fluorescence or NMR), circular dichroism, etc., can also be used, according to techniques known in the art, to obtain thermodynamic parameters of macromolecular solutions. Run in a multiplex fashion these measurements obtain spectroscopic data between binding entities and their substrates that can be interpreted to provide the thermodynamics of the interactions being investigated. One potential drawback for these types of measurements is that interpretation often requires a model of the process, rendering results dependent on accuracy of the model employed.

Multiplex spectroscopic instruments include multiple well micro titer plate systems, multiple cuvette ultraviolet, visible and infrared spectrophotometers, spectropolarimeters and fluorimeters. The power and potential of such instrumentation is that they provide for acquisition of a full thermodyamic profile (enthalpy, entropy and free-energy) of binding interactions, run in parallel multiplex fashion, in a single shot, thereby enabling simultaneous sampling and collection of multiple regions in the temperature dependent thermodynamic trajectory of the interaction space occupied by the binding entities of interest. As described in the examples below, these parallel, multiplex, instruments contain multiple (N) sample chambers or cells (for example N=100 or more). Each sample cell can contain a different macromolecule or mixtures of the same macromolecule in various ratios with a binding entity (a ligand or other macromolecules) present at different concentrations. The temperature dependent thermodyamic transitions of these mixtures are monitored simultaneously in parrallel, multiplex fashion in a single experiment. In such a process, experiments for N different conditions can be performed simultaneously. If collected in conventional serial fashion, the N experiments would have to be performed in successiion, one after the other, drastically increasing the time required to gather the same data.

Multiplex high throughput screening of the thermodynamics of mixtures of two compounds A and B can be performed in various manners. Consider two molecules, A and B, that have binding interactions with one another, e.g., A is the substrate and B is the ligand. The substrate can be, e.g., a protein, nucleic acid molecule, lipid, some combination thereof, or any other material that B binds to. Likewise, B can be a protein molecule, nucleic acid molecule, drug, or any other compound that has binding interactions with A. Using a multiplex instrument, many different iterations of the interactions of B with A can be analyzed. For these examples, it is assume there are at least N sample chambers in the multiplex instrument. Examples of such multiplex instruments might be (but are not limited to) wells of a calorimeter, wells of a microtiter plate, cuvettes of a specrophotometer etc. The multiplex device shall mean that multiple reactions can be run simultaneously in parrallel. A few of the obvious possible interations of how to collect the parallel, multiplex data are given below.

I. In multiplex fashion, A at a constant concentration is placed in each sample chamber. B is then added at different concentrations to each chamber and the resulting signal from each chamber is recorded. In the case where A is a protein or receptor and B is a ligand, the result is a full titration curve recorded in parallel in a single experiment. The output can be analyzed to obtain the thermodynamics of the binding reactions of B for A. In the same manner the full binding space can be sampled in a single experiment by having varying amounts of A present in each sample chamber and adding a constant amount of B to each sample chamber. The savings in time afforded by such a parallel, multiplex strategy is obvious.

II. When the binding space of A with B has been established, i.e. when the range of concentrations and binding constants of A and B have been determined, then in mutiplex fashion, A is present in every chamber at an appropriate constant concentration and a suitable constant concentration of each compound of interest either functionally or structurally related to B, i.e. B1, B2,B3. . . .BN, are added to each sample chamber containing a constant amount of A, and the resulting signal is obtained. Since the binding constant and thermodynamics of the binding of B with A are known, the relative differences observed for each related compound (B 1,B2, B3. . .BN) obtained in the parallel experiment are related directly to differences in binding thermodynamics compared to B. In this way the procedure serves as a relative screen (in the thermodynamic sense) for the binding of compounds related to B that also interact with A.

### ADMET Properties

Many different assays have been developed that measure one or more ADMET properties. Any such assay can be used as part of the methods of the invention, as can data produced by the assays. In some cases, thermodynamic measurements, e.g., of solvent solubility (an absorption and distribution property), can be used to measure one or more ADMET properties. In other cases, non-thermodynamic measurements, e.g., of the diffusion rate or solubility (both reflecting absorption and distribution), of one or more ADMET properties of a compound can be obtained, e.g., using column chromatography (e.g., involving a hydrophobic, anion-exchange, cation-exchange, or size exclusion column mounted on, e.g., an HPLC instrument), a diffusion barrier instrument, or a solubility instrument (e.g., capillary electrophoresis). In still other cases, a biological assay (e.g., an enzyme-based, cell-based, or animal-based assay) can be used to obtain information about ADMET properties such as distribution, metabolism, excretion, and/or toxicity.

Animal-based assays can be particularly useful for determining certain ADMET properties, such as adsorption, distribution, metabolism, excretion, and/or toxicity. Animal assay useful for determining ADMET properties of compounds include, but are not limited to: applying compounds to a surface of an animal, e.g., the skin of a mouse or the eye of a rabbit, and monitoring inflammation of the surface, e.g., vaso-dilation and/or recruitment of blood cells, e.g., white blood cells, e.g., macrophages, neutrophils, etc.; assaying for skin permeation of compounds; intestinal cell permeation assays; blood-brain barrier partitioning assays; and feeding or injecting animals with radiolabeled compounds and following the bodily distribution, excretion, and metabolic breakdown of the compounds.

For reasons of cost and speed, however, it may be preferable to examine ADMET properties such as adsorption, distribution, metabolism and toxicity using a cell-based system or even an enzymatic assay. Example of cell based systems for measuring toxicity include, but are not limited to: Caco-2 cell permeability; adding compounds to water in which there are fairy shrimp or water fleas to test the ability of the compound to cause lethality; the Ames test; and cell-culture systems that measure programmed cell death as a response to differing concentration of a compound. Measures of cell death can be determined, e.g., using vital dyes or fluorescent compounds that react with cellular breakdown products associated with cell death. With regard to metabolism, compounds can be incubated with cells and the chemical alteration of the compound can be monitored by following a radiolabel attached to the compound, or the change or loss of an activity, e.g., fluorescence, associated with the compound.

Enzymatic assays can also be used to measure ADMET properties such as metabolism and toxicity. Such enzymatic assays include, but are not limited to, incubating a chemical compound, e.g., a labeled (e.g., a radiolabeled) or fluroescent compound with a enzyme of interest, e.g., a dehydrogenase or decarboxylase, and monitoring the fate of the chemical compound.

Properties related to one or more ADMET properties include, but are not limited to, solubility, diffusion rate, membrane permeability, and oral bioavailability. An important and specific parameter for oral bioavailability is the transport of the drug across the intestinal epithelial cell barrier. One of the in vitro models, that has been shown to mimic this process, is a Caco-2 cell monolayer. Caco-2 cells, a well-differentiated intestinal cell line derived from human colorectal carcinoma, display many of the morphological and functional properties of the in vivo intestinal epithelial cell barrier. Caco-2 cell models are used with regularity for determination of cellular transport properties, in both industry and academia, as a surrogate marker for in vivo intestinal permeability in humans.

As with measurements relating to therapeutic potency, when evaluating a property related to one or more ADMET properties, it is preferable to use an assay that can be couple with a multi-channel instrument. Multi-channel high throughput instruments are now being developed to determine permeability (an absorption property), solvent solubility (an absorption and distribution property) and selected toxicities of compound libraries. One instrument used for the HTS of compounds with respect to permeation through a nonpolar medium (biological cell wall permeation) as well as for measuring aqueous solubility has been reported. See J. W. McFarland et al. (2001), J. Chem. Inf. Computer Sci., 41(5): 1355-9, the contents of which are incorporated herein by reference. Other instruments that can be used in conjunction with assay intended to evaluate one or more ADMET properties include visual imaging devices (e.g., for counting cells, e.g., stained cells), spectrophotometers, spectropolorimeters, fluorimeters, or calorimeters.

### Construction of Modular Computational Models

Each module of a modular computational model consists of one or more scoring functions, or equations, that relate a measured property, e.g., a therapeutic property, of each compound of a set of compounds with the structure and/or physical properties thereof of the compound. Such scoring functions are often called Quantitative Structure-Activity Relationships (QSARs). QSARs can be used to predict the properties, e.g., therapeutic properties, of compounds that have not been assayed with respect to the particular property predicted by the QSAR. Depending upon the property being measured and the data set used to construct the QSAR, the set of compounds that can be evaluated using the QSAR may be limited or diverse. For example, a QSAR that predicts therapeutic potency and was constructed using a set of training compounds that were highly similar to one another will tend to be limited in terms of the types of compounds that can be evaluated by the QSAR. Alternatively, a QSAR that predicts membrane permeability and was constructed using a structurally diverse set of training compounds may be capable of accurately predicting the membrane permeability properties of a wide range of chemical compounds. Any QSAR, or related type of scoring function, can constitute a module of the invention.

Examples of methods that can be used to construct individual modules of a modular computational model include, but are not limited to, receptor-dependent free energy force field QSAR (FEFF-QSAR), receptor-independent three-dimensional QSAR (3D-QSAR), receptor-dependent or receptor-independent four-dimensional QSAR (4D-QSAR), and membrane interaction QSAR (MI-QSAR).

Receptor-independent 3D-QSAR analysis provides a tool to relate the magnitude of a particular property exhibited by a molecule to one or more structural characteristics and/or physical properties thereof of the molecule. Typically, receptor-independent QSAR is limited in its application to series of chemical analogs for which the dependent (i.e., predicted) property is derived from a set of intramolecular descriptors based upon the assumption that the chemical compounds share a common mechanism of action. As an example, consider thermodynamic data generated in calorimetric experiments. Such data can be employed to calibrate, or scale, an existing force field used in molecular modeling and simulation studies. The component energy terms making up the force field are treated as descriptors (independent variables) in the QSAR paradigm. The dependent variables (the biological activity measures) are the measured thermodynamic properties of the calorimetric experiments being used in the force yield calibration. Regression fitting of the force field energy terms to the each of the thermodynamic property measures of this training set provides a set of regression coefficients that effectively are the calibration factors for the force field. 3D-QSAR methodologies are well known in the art. The scaled force field constitutes a module of a modular computational model that can be applied with a limited range of applicability, but high accuracy, as part of a virtual high throughput screen. In essence such a virtual high throughput screen (VHTS) takes the place of performing actual calorimetric experiments, thus providing the opportunity to explore virtual chemical systems. In the case of exploring ligands binding to a common receptor, virtual sets of ligand analogs can be evaluated in the associated VHTS without having to synthesize any analogs outside of those used to calibrate the force field.

Receptor-dependent, or free energy force field QSAR (FEFF-QSAR), differs from receptor independent 3D-QSAR in that the receptor geometry is known, allowing the free energy force field ligand-receptor binding energy terms to be calculated and used as the independent variables of the QSAR scoring function. The overall methodology is presented in Tokarski and Hopfinger (1997), J. Chem. Inf. Computer Sci. 37:792-811, the contents of which are incorporated herein by reference.

4D-QSAR modules incorporate conformational and alignment freedom into the development of 3D-QSAR modules by performing molecular state ensemble averaging (the fourth dimension) on the training molecules. The descriptors in 3D-QSAR analysis are the grid cell (spatial) occupancy measures of the atoms composing each molecule in the training set produced by sampling conformation and alignment space. Grid cell occupancy descriptors, GCODs, can be generated for a number of different atom types, or as referred to in 4D-QSAR analysis, interaction pharmacophore elements, IPEs. The idea underlying 4D-QSAR analysis is that differences in the activity of molecules are related to differences in the Boltzmann average spatial distribution of molecular shape with respect to the IPEs. A single “active” conformation can be postulated for each compound in the training set, and when combined with the optimal alignment, can be used in additional molecular design applications including receptor independent 3D-QSAR and FEFF-QSAR models. A description of 4D-QSAR models can be found in Duca and Hopfinger (2001), J Chem Inf Comput Sci 41(5):1367-87, the contents of which are incorporated herein by reference.

Membrane-interaction QSAR (MI-QSAR) analysis is a unique method developed to explicitly consider the interaction of a test compound with a model phospholipid membrane in the estimation of cellular permeability coefficients. Many of the ADME properties of a molecule are related to how the molecule interacts with biological membranes. There are also several “mild” toxicity endpoints, like skin and eye irritations, which are also dependent upon how a molecule interacts with cellular membranes. MI-QSAR analysis, like 4D-QSAR analysis developed for the construction of ligand-receptor VHTS, and is unique among modeling and QSAR methods and paradigms in that it is explicitly based on thermodynamics. The thermodynamic basis of MI-QSAR analysis originates from considering the explicit interactions of the test compounds with cellular membranes, solvents and/or other relevant biological media. MI-QSAR analysis simulates the thermodynamics of the molecular process responsible for a particular ADMET property, providing quantitative models of absorption, solvation and toxicological processes. MI-QSAR has been described in Kulkami and Hopfinger (1999), Pharn Res 16(8):1245-53, and Kulkami et al. (2001), Toxicol Sci 59(2):335-45, the contents of which are incorporated herein by reference.

MI-QSAR analysis permits the construction of a VHTS (or module) for an ADMET property from the data determined for a training set using a multi-channel, parallel HTS instrument. The interactive use of multi-channel measurements of ADMET properties and MI-QSAR analysis can, in the initial pass, be used to build a distinct VHTS of each ADMET property measured. Each MI-QSAR module can be used to assay virtual libraries of compounds. The virtual compounds can then be ranked based on their virtual ADMET properties. The highest ranked compounds can then be made and tested in the multi-channel ADMET instrument. The new set of ADMET measurements can then be employed to evolve and refine the existing VHTS, and the entire process repeated until compounds with optimized ADMET properties are realized.

If the ADMET VHTS assays (e.g., MI-QSAR modules) are combined with the biopotency/therapeutic VHTS assays (e.g., 4D-QSAR modules), then if is possible to produce a modular computational model capable of performing global drug-like property optimization. In essence, the substituent sites on a chemical class of compounds that control biopotency are identified as well as the substituent sites that have minimal impact on biopotency. The substituent sites that are not sensitive with respect to biopotency are then selected as the site to optimize the ADMET properties. This process is repeated with respect to substituent sites that are sensitive/insensitive to a specific ADMET property.

Methods of constructing QSAR modules are well known in the art. For example, serial use of partial least squares regression and a genetic function algorithm can be used to identify the best scoring functions for predicting a given therapeutic property without over-fitting the training set data. Genetic function alogorithms tend to identify more than one scoring function that is consistent with the data of the training set, so it is possible that a module will include more than one scoring function and produce more than one predicted value for each member of a plurality of test structures.

In many cases, software is available for use in constructing QSAR models. For example, The Chem21Group, Inc. provides software that can be used to construct any of the modules described herein, e.g., receptor-dependent FEFF-QSAR, receptor-independent 3D-QSAR, receptor-dependent or receptor-independent 4D-QSAR, and MI-QSAR. See, e.g., the 3D-QSAR User's Manual, the 4D-QSAR User's Manual (version 2.0), and the MI-QSAR User's Manual (version 1.0a) from The Chem 21 Group, Inc., the contents of which are incorporated herein by reference.

### Training Compounds/Test Structures

A compound of a training set used to construct a module of a modular computational model can include all or part of a chemical compound, such as a small molecule. As used herein, a small molecule includes, but is not limited to, an organic compound, such as a fatty acid molecule, a sugar molecule, a steroid molecule, a hormone, a peptide, or any derivative or combination thereof. A compound of a training set can further include a chemical compound extracted from an animal, plant, fungus, or single cell organism, such as a bacterium or protist; or a compound that has been synthesized in a laboratory, e.g., by combinatorial chemistry or parallel synthesis.

A training set used in the construction of a module can include a plurality of training compounds, e.g., 5, 10, 20, 30, 40, 50, 75, 100, 125, 150, 200, or more training compounds.

In general, the structures of a plurality of test structures will be related to, e.g., derivatives of, the set of training compounds used to construct the therapeutic potency module. A plurality of test structures can be a set of structures that includes virtual compounds, e.g., compounds wherein only a structural representation, e.g., within a computer data base, is used in the methods of the invention.

### Interaction Partners

As used herein, an interaction partner includes, but is not limited to, a protein, such as a membrane-associated protein, a cytoplasmic protein, or a nuclear protein. Examples of membrane-associated proteins include adhesion receptors (e.g., integrins or cadherins), growth factor signaling receptors (e.g., EGFr, PDGFr, TIE-1 or -2 receptors, insulin receptor, T-cell receptor, etc.), G-protein coupled receptors, glycoproteins (e.g., syndecan or P-, E-, or L-selectin), or transporters (e.g., a Na+ or K+ion transporter or dicarboxylate ion transporter). Examples of cytoplasmic proteins include enzymes (e.g., carboxylases or transferases, e.g., acetyltransferases), ribosomal proteins, kinases (e.g., src, MAPK, PKA, PKC), phosphatases, adapter molecules (e.g., IRS-1, Shc, GRB2, SOS), GTPases (e.g., ras, rac, rho, cdc42) or an ATPase. Examples of nuclear proteins include transcription factors (e.g., TFIID), polymerases, or chromatin-associated proteins (e.g., histones). The interaction partner can be a lipid, e.g., a modified lipid, e.g., phosphatidyl inositol 4, 5-phosphate or a similar lipid involved in signaling pathways, e.g., diacyl glycerol. The interaction partner can also include a nucleic acid molecule, e.g., DNA or RNA. The interaction partner can be a supramolecular structure, e.g., a multi-subunit protein complex, a protein-DNA or protein-RNA complex, a lipid membrane (e.g., a micelle, a lipid monolayer, a lipid bilayer, or any cellular or in vitro membrane having properties identical or consistent with biological barriers), or any combination thereof. In addition, the interaction partner can be a cell, e.g., a mammalian cell, an insect cell, a fungal cell, a bacterium, or a protist.

### Evaluating the Screened Structures

After screening a set of structures with respect to one or more pharmaceutical properties, it will typically be useful to evaluate the predicted screening results so that compounds having desirable pharmaceutical properties can be identified. Such evaluation can easily be accomplished by either comparing the predicted properties or measurements with a reference value or ranking the entire set of structures with respect to their predicted properties. Comparing the predicted properties with a reference value, e.g., a reference value that is associated with a desirable pharmaceutical property, can provide an unbiased assessment of the structures with respect to that property. It may be useful, e.g., to evaluate therapeutic potency relative to a reference value, as a structure that does not have a minimum therapeutic potency will probably not be pursued further. Alternatively, it may be useful to know which structures fell below a certain threshold value for a particular property and their may be a structural relationship between structures that have a poor therapeutic property. On the other hand, ranking compounds relative to one another can also be useful. For example, in a subset of compounds that score above a certain threshold for pharmaceutical potency, it may be useful to know how they rank relative to one another with regard to a distinct pharmaceutical property, such as an ADMET property. Such a process can allow structures that are globally optimized to be identified.

### Data Structures

After screening a plurality of structures for one or more desirable properties, it may be useful to maintain a record of the results of the screen. Such records could be useful, for example, in comparing the relative performance of different modular computational models, e.g., for reviewing how an increase in the size of the training set effects the performance of one or more modules in the modular computational model. Thus, the invention is believed to encompass any data structure containing at least some property predictions that may arise from performing the methods of the invention. For example, the data structure, which may be a database, e.g., a computer database, can include all of the predications, or just a subset of predictions, e.g., best and/or worst scoring structures and their predicted properties, arising from using the methods of the invention to evaluate a plurality of test structures, such as a library. The resulting data structure could be, e.g., computer readable, and could have a plurality, e.g., 10, 50, 100, 1,000, 5,000, 10,000, or more stored predictions.

### EXAMPLES

### Example 1:

The force field scaling/calibration approach has been successfully applied to develop ligand-receptor force fields specific to a given enzyme and a given chemical class of inhibitors. A training set of glucose analog inhibitors of glycogen phosphorylase, GP, was used to develop a FEFF 3D-QSAR force field for this system. See P. Venkatarangan, A. J. Hopfinger (1999), J. Med. Chem. 42: 2169-2179, the contents of which are incorporated herein by reference. The free energy of glucose analog—GP binding, ΔG, as an example, is given by:

ΔG=−0.09EL(LL)−0.14ELR,vdw−0.05DER,str(RR)−0.99ELR,vdw(LL)+0.08

N=39 R²=0.88 Q²=0.80

where:

N is the number of observations (training set compounds);

R is the correlation coefficient;

Q is the leave-one-out cross-validation coefficient;

EL(LL) is the un-scaled force field minimum conformational energy of the isolated ligand;

ELR,vdw the un-scaled force field ligand-receptor interaction van der Waals energy associated with the minimum energy complex;

DER,str(RR) the change in the bond stretching energy of the receptor upon ligand complexing to the receptor; and

ELR,vdw(LL) the van der Waals energy of the ligand when bound to the Receptor.

### Example 2

In another application of FEFF 3D-QSAR a training set of peptido-mimetic renin inhibitors was used to develop a scaled force field to compute the free energy of binding of virtual peptido-mimetic inhibitors to renin. The free energy FEFF 3D-QSAR model, that is the scaled force field, found in this study for the binding free energy (ΔG) is:

ΔG=0.06EL(LL)−0.05DEsolv+7.74

N=12, R²=0.85, Q²=0.77

where:

EL(LL), N, R, and Q are the same as defined above for the glucose analog inhibitor-GP system; and

DEsolv is the change in un-scaled force field aqueous solvation energy of ligand-receptor binding.

Corresponding FEFF 3D-QSAR scaled force field equations have also been constructed for ΔH and ΔS for each of these two inhibitor enzyme systems. Thus, the parent force field, which in both these examples is an AMBER-1 force field (see Weiner et al. (1986), J Comput Chem 7:230-52), has been scaled against the measured thermodynamic properties of binding of the training sets to provide virtual thermodynamic binding screens. The virtual screens, in turn, are then used to perform virtual screening of libraries of virtual inhibitors. The net achievement of this FEFF 3D-QSAR approach is to rapidly, and reliably, screen and rank hypothetical inhibitors for further consideration in terms of actual synthesis and testing.

The force field can be systematically decomposed into an increasing number of descriptors that, in composite additive-difference format, make up the mathematical representation of the force field. It is possible, for example, to go from a small set of descriptors consisting of only the net changes in the energy terms due to ligand-receptor binding all the way to a very large descriptor set including individual pair-wise atomic interactions. This can be both good and bad. It can be good in that a very large number of descriptors are available to develop a scaled force field that very precisely fits the training set data. It can be bad in that the force field may over fit the data and/or not be the best functional representation. Fortunately, there are algorithms and methods to explore and solve both these types of problems. A combination of partial least-square, PLS, regression and application of a genetic algorithm permits the optimized force field to be determined in terms of data fit, robustness and consistency.

The thermodynamic data binding data used in the peptido-mimetic renin FEFF3D-QSAR study illustrates the additional binding information that comes with thermodynamic studies as compared to current in vitro biological screens. Table 1 lists compounds of the training set used to calibrate the force field, while Table 2 lists thermodynamic measurements obtained for the renin inhibitors of Table 1.

TABLE 1
Renin inhibitor structures used to
construct the FEFF 3D-QSAR module

| Compound | Structure |
|---|---|
| U80631E | Ac-phe-his-leu-y[CH(OH)CH2]val-ile-NH2 |
| U77646E | Ac-pro-phe-his-leu-Y[CH(OH)CH2]val-ile-NH2 |
| U77647E | Ac-D-pro-phe-his-leu-Y[CH(OH)CH2]val-ile-NH2 |
| U73777E | Ac-phe-his-phe-Y[CH2NH]phe-NH2 |
| U71909E | Ac-pro-phe-his-phe-Y[CH2NH]phe-NH2 |
| U77451E | Ac-pro-phe-his-phe-Y[CH2NH]phe-Mba |
| U72407E | Ac-phe-his-sta-ile-NH2 |
| U72408E | Ac-pro-phe-his-sta-ile-NH2 |
| U72409E | Ac-his-pro-phe-his-sta-ile-NH2 |
| U77455E | Iva-his-pro-phe-his-sta-ile-phe-NH2 |

TABLE II
Thermodynamic properties of the renin inhibitors

| Compound | Kd μm | −ΔH kcal/mole | −ΔS kcal/mole | −ΔG kcal/mole |
|---|---|---|---|---|
| U80631E | 0.37 | 14.28 | 75.7 | 9.2 |
| U77646E | 0.0054 | 28.75 | 131.1 | 11.5 |
| U77647E | 0.0013 | 20.33 | 105.5 | 12.4 |
| U73777E | 0.22 | 14.20 | 76.3 | 9.4 |
| U71909E | 0.029 | 13.70 | 78.4 | 10.6 |
| U77451E | 0.0025 | 26.70 | 125.3 | 12.2 |
| U72407E | 0.204 | 26.10 | 114.8 | 9.5 |
| U72408E | 0.098 | 14.69 | 79.6 | 9.9 |
| U72409E | 0.023 | 22.63 | 108.0 | 10.8 |
| U77455E | 0.0017 | 21.36 | 108.9 | 12.4 |

Taken from Epps et al. (1990), Med Chem., 2080-2086, the contents of which are incorporated herein by reference.

The data in Table 2 demonstrates that important additional information comes from the invention. The normal first pass assessment of a ligand as an effective inhibitor of an enzyme, and its potential as a drug candidate, comes from the measurement of Kd, or a near equivalent measure reflecting the inhibition potency of the test ligand. This initial test serves as a “Yes or No” answer as to whether or not to further consider evaluation of a ligand as a drug candidate. The pair of compounds U73777E (Kd=0.22, ΔG=9.4) and U72407E (Kd=0.203, ΔG=9.5) would be judged to be about identical in ligand-receptor binding based solely on their measured Kd and ΔG values. However, the specific binding of U72407E, as measured by ΔH (26.10) is considerably higher than that of U73777E (14.20). This same situation is seen in comparing compounds U71909E and U72409E.

The enthalpy of binding, ΔH, is almost never experimentally measured in current ligand-recpetor binding screens including HTS methods. On the other hand, it is the ΔH of binding which is the property approximately computed using computational methods of predicting ligand-recpetor binding. Thus, there is a major inconsistency inherent to comparing current experimental and computational measurements of ligand-receptor binding thermodynamics which can be overcome by application of the invention. But perhaps more important, ΔH is a direct measure of the binding specificity. The more specific the binding of a ligand to a particular receptor, the less is the chance of specific binding to another receptor and the corresponding expression of toxicity by the ligand. Current experimental; methods of evaluating ligand-receptor binding do not measure ΔH and, therefore, give a limited assessment of ligand interaction specificity. The invention provides a means of obtaining the most information regarding ligand-receptor binding specificity by determining the enthalpy of ligand-receptor binding.

### Example 3

A dependent variable that can be used in MI-QSAR analysis is the Caco-2 cell permeability coefficient, Pcaco-2. Yazdanian and coworkers (see Yazdanian et al. (1998), Pharmaceutical Research 15:1490-94, the contents of which are incorporated herein by reference) performed permeability experiments on a data set of 38 structurally and chemically diverse drugs ranging in molecular weight from 60 to 515 amu and varying in net charge at pH 7.4.

Table 3 contains the Pcaco-2 values for 30 structurally diverse drugs used as the training set of compounds and 8 drugs used as a test set.

TABLE 3
The Molecular Weight, Caco-2 Permeability Coefficient,
and Corresponding Percent of Drug Absorbed for the
Drugs of the Training and Test Sets

| | | Permeability × | |
|---|---|---|---|
| Drug | MW | 10⁶ (cm/sec) | % Absorbed |
| TRAINING SET | | | |
| Diazepam | 284.74 | 33.40 | 100 |
| Caffeine | 194.19 | 30.80 | 100 |
| Phenytoin | 252.27 | 26.70 | 90 |
| Alprenolol | 249.35 | 25.30 | 93 |
| Testosterone | 288.43 | 24.90 | 100 |
| Phencyclidine | 243.39 | 24.70 | — |
| Desipramine | 266.39 | 24.20 | 95 |
| Metoprolol | 267.37 | 23.70 | 95 |
| Progesterone | 314.47 | 23.70 | — |
| Salicylic acid | 138.12 | 22.00 | 100 |
| Clonidine | 230.10 | 21.80 | 100 |
| Corticosterone | 346.47 | 21.20 | 100 |
| Indomethacin | 357.79 | 20.40 | 100 |
| Chlorpromazine | 318.86 | 19.90 | 90 |
| Nicotine | 162.23 | 19.40 | 100 |
| Estradiol | 272.39 | 16.90 | — |
| Pindolol | 248.32 | 16.70 | 95 |
| Hydrocortisone | 362.47 | 14.00 | 89 |
| Timolol | 316.42 | 12.80 | 72 |
| Dexamethasone | 392.47 | 12.20 | 100 |
| Scopolamine | 303.36 | 11.80 | 100 |
| Dopamine | 153.18 | 9.33 | — |
| Labetalol | 328.41 | 9.31 | 90 |
| Bremazocine | 315.45 | 8.02 | — |
| Nadolol | 309.40 | 3.88 | — |
| Atenolol | 266.34 | 0.53 | 50 |
| Terbutaline | 225.29 | 0.47 | 73 |
| Ganciclovir | 255.23 | 0.38 | 3 |
| Sulfasalazine | 398.39 | 0.30 | 13 |
| Acyclovir | 225.21 | 0.25 | 20 |
| TEST SET | | | |
| Aminopyrine | 231.3 | 36.5 | 100 |
| Propranolol | 259.35 | 21.80 | 90 |
| Warfarin | 308.33 | 21.10 | 98 |
| Meloxicam | 351.39 | 19.50 | 90 |
| Zidovudine | 267.24 | 6.93 | 100 |
| Urea | 60.06 | 4.56 | — |
| Sucrose | 342.30 | 1.71 | — |
| Mannitol | 182.17 | 0.38 | 16 |

The construction of the training and test sets was accomplished by insisting that members of the test set be representative of all members of the training set in terms of the ranges of Pcaco-2 values, molecular weights and structural and chemical diversities. Table 3 also contains a composite summary of the “% absorbed” of many of the drugs in the table. These data were compiled by search of the literature. It can be seen from a comparison of the Pcaco-2 and “% absorbed” that Pcaco-2 is indeed indicative of in vivo drug absorption/uptake. The 30 compounds of the training set have been incorporated into the MI-QSAR analysis to build a Caco2 cell permeation VHTS in a manner that simulates the output from a multi-channel HTS ADMET property measurement instrument.

The best MI-QSAR models for Caco-2 cell permeability realized by considering the combination of general intramolecular solute, intermolecular dissolution/solvation-solute and intermolecular membrane-solute descriptors are presented as a function of the number of terms, that is descriptors, included in a given MI-QSAR model:

1 term model:

Pcaco-2=37.39+0.73F(H2O)

N=30, R²=0.75, Q²=0.71

2 term model:

Pcaco-2=30.58+0.54F(H2O)+0.07ΔETT(hb)

N=30, R²=0.78, Q²=0.72

3 term model:

Pcaco-2=31.87+0.72F(H2O)+0.07ΔETT(hb)−0.26ESS(hb)

N=30, R²=0.80, Q^{2 =}0.74

4 term model:

Pcaco-2=−14.62+0.71F(H2O)+0.07ΔETT(hb)−0.26ESS(hb)+0.06ETT(14)

N=30, R²=0.82, Q²=0.75

5 term model:

Pcaco-2=−16.16+0.73F(H2O)+0.06ΔETT(hb)−0.25ESS(hb)+0.07ETT(14)−0.12ETT(tor)

N=30, R²=0.83, Q²=0.74

6 term model:

Pcaco-2=−40.50+0.65F(H2O)+0.06ΔETT(hb)−0.19ESS(hb)+0.10ETT(14)−0.03ETT(tor)−5.61 χ3

N=30, R²=0.86, Q²=0.77

where N is the number of compounds, R² is the coefficient of determination, and Q² is the cross-validated coefficient of determination.

The descriptors found in the best MI-QSAR models are as follows:

1) F(H2O) is the aqueous solvation free energy;

2)χ3 is a Kier-Hall topological index;

3) ESS(hb) is the intramolecular hydrogen bonding energy of the solute molecule when it is in the lowest membrane-solute interaction state within the membrane;

4) ΔETT(hb) is the change in the hydrogen bonding energy of the entire membrane-solute for the solute re-located from free-space to the position corresponding to the lowest solute - membrane interaction energy state of the model system;

5) ETT(14) is the 1,4-Van der Waals plus electrostatic interaction energy of the entire membrane-solute system for the solute located at the position corresponding to the lowest solute membrane interaction energy state of the model system. The range in values of this descriptor over the training and test sets is 770-920 kcals/mole, a very large set of energies. However, there are over 700 torsion angles associated with ETT(14). Thus, the average ETT(1,4) per torsion angle is only about 1.1 to 1.3 kcals/mole; and

6) ETT(tor) is the torsion energy of the entire membrane-solute system for the solute located at the position corresponding to the lowest solute-membrane interaction energy state of the model system. This descriptor is also large in energy having a range of values of 150-230 kcals/mole across the training and test sets of compounds. Again, for the more than 700 torsion angles associated with this descriptor, the average value of ETT(tor) per torsion angle is only 0.20 to 0.33 kcal/mole.

TABLE 4
The general intramolecular solute descriptors
used in the trial MI-QSAR descriptor pool.

| | |
|---|---|
| HOMO | (Highest occupied molecular orbital energy) |
| LUMO | (Lowest occupied molecular orbital energy) |
| Dp | (Dipole moment) |
| Vm | (Molecular Volume) |
| SA | (Molecular surface area) |
| Ds | (Density) |
| MW | (Molecular weight) |
| MR | (Molecular refractivity) |
| N (hba) | (Number of hydrogen bond acceptors) |
| N (hbd) | (Number of hydrogen bond donors) |
| N (B) | (Number of rotatable bonds) |
| JSSA (X) | (Jurs-Stanton surface area descriptors) |
| Chi-N, Kappa-M | (Kier & Hall topological descriptors) |
| Rg | (Radius of Gyration) |
| PM | (Principle moment of inertia) |
| Se | (Conformational entropy) |
| Q (I) | (Partial atomic charge densities) |

TABLE 5
The intermolecular interaction descriptors in the trial
MI-QSAR descriptor pool. Part A includes the membrane-solute
interaction descriptors, and Part B lists the intermolecular
dissolution and solvation descriptors of the solute.
Part A

| The membrane-solute | |
|---|---|
| descriptors—Symbols | Description of the membrane-solute descriptors |
| <F(total)> | Average total free energy of interaction of the |
| | solute and membrane |
| <E(total)> | Average total interaction energy of the solute |
| | and membrane |
| E_{INTER} (total) | Interaction energy between the solute and the |
| | membrane at the total intermolecular system |
| | minimum potential energy |
| E_{XY}(Z) | Z = 1,4-nonbonded, general Van der Waal, |
| | electrostatic, hydrogen bonding, torsion and |
| | combinations thereof energies at the total |
| | intermolecular system minimum potential |
| | energy. |
| | X, Y can be the solute, S, and/or membrane, M |
| ΔE_{XY}(Z) | Change in the Z = 1,4-nonbonded, general Van |
| | der Waal, electrostatic, hydrogen bonding, |
| | torsion and combinations thereof energies due |
| | to the uptake of the solute to the total |
| | intermolecular system minimum potential |
| | energy. |
| | X, Y can be the solute, S, and/or membrane, M |
| E_{TT}(Z) | Z = 1,4-nonbonded, general Van der Waal, |
| | electrostatic, hydrogen bonding, torsion and |
| | combinations thereof energies of the total |
| | [solute and membrane model] intermolecular |
| | minimum potential energy |
| ΔE_{TT}(Z) | Change in the Z = 1,4-nonbonded, general Van |
| | der Waal, electrostatic, hydrogen bonding and |
| | combinations thereof of the total [solute and |
| | membrane model] intermolecular minimum |
| | potential energy |
| ΔS | Change in entropy of the membrane due to the |
| | uptake of the solute |
| S | Absolute entropy of the solute-membrane |
| | system |
| Δρ | Change in density of the model membrane due |
| | to the permeating solute |
| <d> | Average depth of the solute molecule from the |
| | membrane surface |

Part B

| Dissolution and solvation— | Description of the dissolution/ |
|---|---|
| solute descriptors—Symbols | solvation—solute descriptors |
| F(H2O) | The aqueous solvation free energy |
| F(OCT) | The 1-octanol solvation free energy |
| Log(P) | The 1-octanol/water partition coefficient |
| E(coh) | The cohesive packing energy of the solute |
| | molecules |
| T_{M} | The hypothetical crystal-melt transition |
| | temperature of the solute |
| T_{G} | The hypothetical glass transition |
| | temperature of the solute |

The values of the six descriptors found in the 1- to 6-tern MI-QSAR models for each compound in the training and test sets are given in Table 6. Using the 3- through 6-term MI-QSAR models, the observed and predicted Caco-2 cell permeation coefficients of the test and training set compounds are listed in Table 7. Clonidine, metoprolol, corticosterone and aminopyrine are observed to permeate better than predicted by each of the MI-QSAR models, while nicotine and progesterone have a lower permeation coefficient than are predicted by any of the models. Nevertheless, none of the compounds in either the training or test sets are outliers for the 3-through 6-term MI-QSAR models. R₂, for both the training and full sets, increases with increasing number of descriptor terms. However, Q² dips in value for the 5-term model, perhaps suggesting over-fitting is being approached with the 5- and 6-term models for the training set.

TABLE 6
The values of the six significant MI-QSAR descriptors

| Structure | | | | | | |
|---|---|---|---|---|---|---|
| Name | E_{TT}(tor) | E_{TT}(14) | E_{SS}(hb) | ΔE_{TT}(hb) | χ3 | FH20 |
| diazepam | 196.9 | 847.4 | 0.0 | 0.0 | 0.0 | 6.87 |
| caffeine | 180.3 | 792.0 | 0.0 | 0.0 | 0.0 | 5.47 |
| phenytoin | 166.2 | 826.4 | −1.8 | −23.6 | 0.0 | −11.89 |
| alprenolol | 167.7 | 830.9 | −8.9 | −6.0 | 0.0 | −18.99 |
| testosterone | 168.2 | 833.9 | 0.0 | −18.0 | 0.0 | −9.04 |
| phencyclidine | 212.4 | 808.9 | 0.0 | 0.0 | 0.0 | −3.67 |
| desipramine | 150.3 | 806.0 | −0.9 | −7.2 | 0.0 | −11.66 |
| metoprolol | 169.4 | 820.2 | −6.0 | −13.3 | 0.0 | −22.16 |
| progesterone | 185.3 | 823.1 | 0.0 | 0.0 | 0.0 | −0.07 |
| salicylicacid | 173.8 | 809.9 | −10.5 | −7.6 | 0.0 | −16.13 |
| clonidine | 215.3 | 798.9 | 0.0 | −40.8 | 0.0 | −15.97 |
| corticosterone | 208.3 | 806.4 | −7.1 | −48.6 | 0.0 | −18.74 |
| Indomethacin | 188.1 | 855.6 | −1.4 | −6.8 | 0.0 | −18.42 |
| chlorpromazine | 158.4 | 794.1 | 0.0 | 0.0 | 0.0 | −10.00 |
| nicotine | 203.7 | 800.1 | 0.0 | 0.0 | 0.0 | −6.34 |
| estradiol | 163.7 | 815.5 | 0.0 | −39.4 | 0.0 | −20.15 |
| pindolol | 169.6 | 829.9 | −6.5 | −61.7 | 0.0 | −26.24 |
| hydrocortisone | 160.4 | 825.6 | −15.6 | −51.0 | 0.0 | −28.04 |
| timolol | 178.7 | 808.7 | −15.1 | −21.7 | 0.0 | −30.43 |
| dexamethasone | 230.8 | 877.4 | −14.7 | −64.4 | 0.0 | −27.93 |
| scopolamine | 185.1 | 859.2 | −6.4 | −7.6 | 1.4 | −22.16 |
| dopamine | 201.1 | 809.4 | −5.7 | −25.4 | 0.0 | −28.43 |
| labetalol | 149.5 | 792.9 | −25.9 | −45.3 | 0.0 | −36.37 |
| bremazocine | 216.6 | 836.4 | −3.2 | −48.3 | 1.5 | −22.57 |
| nadolol | 187.2 | 823.4 | −18.3 | −50.4 | 0.0 | −38.74 |
| ntenolol | 168.9 | 783.4 | −7.5 | −123.0 | 0.0 | −28.82 |
| terbutaline | 172.0 | 770.3 | −13.8 | −54.9 | 0.0 | −33.38 |
| ganciclovir | 204.1 | 783.3 | −35.7 | −126.0 | 0.0 | −43.23 |
| sulfasalazine | 164.3 | 766.8 | −7.5 | −22.8 | 0.0 | −37.92 |
| acyclovir | 183.8 | 805.9 | −16.6 | −127.4 | 0.0 | −34.13 |

Test Set

| | | | | | | |
|---|---|---|---|---|---|---|
| aminopyrine | 225.58 | 859.5 | 0 | 0 | 0 | 8.72 |
| propranolol | 171.17 | 805.93 | −6.55 | −46.43 | 0 | −20.89 |
| warfarine | 203.19 | 859.62 | −3.49 | 5.94 | 0 | −18.10 |
| meloxicam | 217.59 | 917.53 | −39.16 | −20.45 | 0 | −26.24 |
| zidovudine | 187.44 | 785.1 | −8.4 | −31.26 | 0 | −26.08 |
| urea | 203.81 | 816.94 | 0 | −186.09 | 0 | −18.60 |
| mannitol | 186.59 | 838.82 | −48.12 | −102.16 | 0 | −53.67 |
| sucrose | 205.11 | 866.78 | −141.11 | −132.76 | 0 | −83.58 |

TABLE 7
Observed and predicted Caco-2 permeability coefficients for the 3- to
6-term MI-QSAR models.
Training Set

| Structure Name | Obs. P_{caco-2}x10⁶ | 3 Term | 4 Term | 5 Term | 6 Term |
|---|---|---|---|---|---|
| Diazepam | 33.4 | 26.89 | 27.84 | 27.99 | 29.25 |
| Caffeine | 30.8 | 27.91 | 25.73 | 25.99 | 25.43 |
| Phenytoin | 26.7 | 21.97 | 21.95 | 23.50 | 23.82 |
| Alprenolol | 25.3 | 19.99 | 20.26 | 21.40 | 22.03 |
| Testosterone | 24.9 | 23.98 | 24.30 | 25.87 | 26.35 |
| Phencyclidine | 24.7 | 29.22 | 27.95 | 26.86 | 27.15 |
| Desipramine | 24.2 | 23.13 | 21.88 | 23.78 | 23.44 |
| Metoprolol | 23.7 | 16.38 | 16.15 | 17.04 | 17.87 |
| Progesterone | 23.7 | 31.82 | 31.29 | 31.88 | 31.75 |
| Salicylicacid | 22 | 22.38 | 21.43 | 22.06 | 21.90 |
| Clonidine | 21.8 | 17.27 | 15.87 | 14.58 | 15.48 |
| Corticosterone | 21.2 | 16.53 | 15.64 | 14.82 | 15.44 |
| Indomethacin | 20.4 | 18.40 | 20.04 | 20.51 | 22.63 |
| Chlorpromazine | 19.9 | 24.63 | 22.65 | 23.94 | 23.41 |
| Nicotine | 19.4 | 27.28 | 25.57 | 24.73 | 24.87 |
| Estradiol | 16.9 | 14.34 | 13.94 | 15.39 | 16.14 |
| Pindolol | 16.7 | 9.97 | 10.60 | 12.02 | 13.13 |
| Hydrocortisone | 14 | 11.83 | 12.20 | 13.87 | 14.24 |
| Timolol | 12.8 | 12.15 | 11.47 | 11.58 | 12.25 |
| Dexamethasone | 12.2 | 10.68 | 14.01 | 12.95 | 15.89 |
| Scopolamine | 11.8 | 16.91 | 18.83 | 19.41 | 13.54 |
| Dopamine | 9.33 | 10.87 | 10.21 | 9.28 | 10.88 |
| Labetalol | 9.31 | 8.90 | 7.56 | 9.03 | 8.33 |
| Bremazocine | 8.02 | 12.77 | 13.63 | 12.70 | 6.39 |
| Nadolol | 3.88 | 4.83 | 5.26 | 5.22 | 6.71 |
| Ntenolol | 0.53 | 3.78 | 2.17 | 3.56 | 3.29 |
| Terbutaline | 0.47 | 7.18 | 4.57 | 4.76 | 4.50 |
| Ganciclovir | 0.38 | 0.44 | −0.90 | −1.69 | −2.23 |
| Sulfasalazine | 0.3 | 4.66 | 1.77 | 1.83 | 2.36 |
| Acyclovir | 0.25 | 1.95 | 1.72 | 2.56 | 2.88 |

Test Set

| Structure Name | Observed BA | 3 Term | 4 Term | 5 Term | 6 Term |
|---|---|---|---|---|---|
| Aminopyrine | 36.5 | 25.56 | 27.20 | 26.01 | 28.25 |
| Propranolol | 21.8 | 14.98 | 14.10 | 15.09 | 15.26 |
| Warfarine | 21.1 | 19.23 | 21.08 | 20.84 | 23.16 |
| Meloxicam | 19.5 | 21.53 | 26.84 | 26.60 | 28.61 |
| Zidovudine | 6.93 | 12.84 | 10.80 | 10.36 | 10.67 |
| Urea | 4.56 | 4.51 | 4.91 | 5.95 | 6.53 |
| Mannitol | 0.38 | −2.10 | −0.27 | 0.07 | 0.70 |
| Sucrose | 1.71 | −1.87 | 2.22 | 1.50 | −1.39 |

It appears from an analysis of the six scoring functions that FH2O in the one-term model accounts for much of the variance of Pcaco-2 across the training set. Nevertheless, the descriptors of the 2-through 6-term MI-QSAR models are all membrane-solute interaction properties and, therefore, judged as being important in characterizing the mechanism of solute-membrane permeation. A composite analysis of all the MI-QSAR scoring functions suggests that the 3-term MI-QSAR model captures the essential features of the postulated mechanism responsible for solute-membrane permeability as represented by Pcaco-2 values. The 3-term model does not represent a distinctly large statistical improvement over the 2-term model, but rather includes descriptors indicative of each of the three components of the postulated mechanism of permeation.

The descriptors of the 4-, 5-, and 6-term MI-QSAR scoring functions successively refine the 3-term model, fitting to the training set. The possible significance of the descriptors added in the 4-to 6-term MI-QSAR scoring functions to further revealing the essential mechanism of Caco-2 cell permeation can only be ascertained by consideration of an expanded training set. The interpretation that the 4-, 5-, and 6-term MI-QSAR models are successive refinements of the “basic” 3-term MI-QSAR model is also supported by the mathematical forms of the MI-QSAR models. The [n+l]-term MI-QSAR model can be viewed as essentially the [n]-term model with one new additional descriptor. The regression coefficients of corresponding descriptor terms across all of the MI-QSAR models are remarkably similar to one another, which indicates their respective roles in predicting Pcaco-2 are about the same in each MI-QSAR model irrespective of the number of descriptor terms in the model.

A test set of eight solute compounds was constructed from the parent Caco-2 cell permeation coefficient data set as one way to attempt to validate the MI-QSAR models. The drugs (solute molecules) of the test set were selected so as to span the entire range in Caco-2 cell permeability for the composite training set. The observed and predicted Pcaco-2 values for this test set are given at the bottom of Table 7. There are no outliers, but aminopyrine and propanol, compounds 1 and 2 of the test set, are predicted to have a lower permeability coefficients than observed. Conversely, meloxican has a higher observed Pcaco-2 value than is computed from any of the MI-QSAR models.

The aqueous solvation free energy, F(H2O) has been shown to correlate to aqueous solubility as would be expected. Increasingly negative F(H2O) values corresponds to increasing aqueous solubility of a solute. In the Pcaco-2 MI-QSAR models it is seen that F(H2O) is positively correlated to Pcaco-2. This relationship indicates that water soluble compounds will have lower permeability coefficients than hydrophobic compounds. This observation is similar to those found in the literature where Log P has been shown to have a relationship to Caco-2 cell permeability. An increase in Log P, reflecting an increase in lipophilicity, often corresponds to an increase in Caco-2 cell permeability. However, the relationship between Log P and Caco-2 cell permeability is not well defined. Some researchers report a sigmoidal relationship while others report a poor linear relationship. A significant linear relationship between F(H2O) and Pcaco-2 is seen in the MI-QSAR models reported here starting with the one-term MI-QSAR model. Our interpretation of this relationship is that the other descriptors of the MI-QSAR models, which focus on explicit membrane-solute interactions, are not considered in the models/relationships of other workers. Hence, the models developed by other workers necessarily contain “noise” in the Log P—Caco-2 cell permeation comparisons and relationships.

ΔETT(hb) is the difference in the total hydrogen bond energy of the solute in the membrane minus the solute being in free space and the membrane by itself. No hydrogen bonding can occur within, or between, DMPC molecules. Thus, the hydrogen bond energy of the membrane by itself is zero and:

ΔETT(hb)=ESS(hb)−E′SS(hb)+EMS(hb) (1)

where E′SS(hb) is the intramolecular solute hydrogen bonding energy for the solute in free-space. In the MI-QSAR models containing ΔETT(hb) the regression coefficients of this descriptor term are positive and about equal. Thus, if intramolecular hydrogen bonding of the solute decreases upon uptake into the membrane, ESS(hb), and/or increases for the solute in free space, E′SS(hb), the permeation coefficient of the solute will increase. A decrease in intramolecular solute hydrogen bonding should correspond to an increase in the conformational flexibility of the solute. Solute conformational flexibility within the membrane is very important for high permeability as other MI-QSAR model descriptors, see below, also indicate. However, while AESS(hb) is the preferred descriptor with FH2O in a 2-term MI-QSAR model, ESS(hb) is the next preferred descriptor and is found in the best 3-term MI-QSAR model. Thus, the terms:

{aΔETT(hb)−bESS(hb)}={[a-b]ESS(hb)−aE′SS(hb)−aEMS(hb)} (2)

are always present indicating the most important contribution of the 3-term MI-QSAR model to refining the 2-term MI-QSAR model is to correct the statistical weighting of ESS(hb) in the 2-term model since it is inherent to the ΔETT(hb) descriptor.

If intramolecular hydrogen bonding of the solute decreases upon uptake into the membrane, solute-membrane hydrogen bonding will likely increase. According to equation (1), and the MI-QSAR models, an increase in solute-membrane hydrogen bonding will diminish solute permeability. Thus, the joint interpretation of ΔETT(hb) and ESS(hb) in the MI-QSAR models is that they capture the balance of hydrogen bonding of the solute with itself in and out of the membrane, and with the DMPC molecules of the membrane, that is at play in the solute-membrane permeation process.

Solute and DMPC conformational flexibility is represented by ETT(14) in the 4-, 5-, and 6-term scoring functions and ETT(tor) in the 5- and 6-term scoring functions. ETT(14) is the Van der Waals and electrostatic energies associated with each set of atoms separated exactly, and only, by one torsion angle in the solute molecule and all the DMPC molecules of the model membrane. This contribution to the total conformational energy measures the composite rigidity of an average torsion rotation of the entire solute-membrane system. As ETT(14) increases the molecules of the membrane-solute system, on average, are moving away from minimum energy conformer states and exploring more conformational states That is, the molecules are expressing greater flexibility. This greater flexibility results in a higher permeation coefficient of the solute molecule based on the positive regression coefficients for ETT(14) in the 4-, 5- and 6-term scoring functions. Presumably, an increase in conformational flexibility of the membrane-solute system makes it easier for the solute to navigate through the membrane.

ETT(tor) is always positive in energy value and measures the force field torsional potential energy for the bonds about which rotations occur in the membrane-solute system. The greater the value of ETT(tor), the greater the average flexibility of the membrane -solute system with regard to torsion angle flexibility for the same reasons as expressed for ETT(14). However, the regression coefficient for this descriptor is negative in the 5- and 6-term scoring functions, and consequently, Pcaco-2 is predicted to decrease as ETT(tor) increases. Thus, it would seem that ETT(tor) is acting as a refinement term to ETT(14) in the 5- and 6-term scoring functions in the same way that ESS(hb) “refines” ΔETT(hb) in the 4-, 5-, and 6-term scoring functions.

The joint roles of ESS(hb) and ΔETT(hb), as expressed by eq.(2), and their influence on solute permeability, may be reflected in the preferred MDS “docking” locations of the solutes within the model-membrane. Solutes having low permeation coefficients tend to dock near the polar heads of the model membrane monolayer. These solutes generally have strong intermolecular hydrogen bond and/or electrostatic interactions with head groups and/or the C═O groups of the phospholipids. Solutes with high permeability coefficients either have no preferred docking sites in the monolayer, or preferentially locate in the tail regions of the DMPC phospholipids. These solutes are flexible and/or have limited hydrogen bond and/or electrostatic interactions with the membrane.

It has been shown in past studies that Caco-2 cell permeability correlates with the number of hydrogen bond donor, or acceptor, groups in the solute molecule. The fewer the number of donors and/or acceptors, then the better the permeability of the solute. Still, there are compounds that have several hydrogen bonding sites, but at the same time, have high permeation coefficients. One explanation for this apparent conflict, which is consistent with the presence of F(H2O), ΔETT(hb) and ESS(hb) in the MI-QSAR models comes from the hypothesis of Stein. This hypothesis asserts that the rate-limiting step in the transport of a polar solute across a cell membrane is aqueous desolvation. For a polar solute to transverse a cell membrane, the hydrogen bonds formed with water molecules must be broken. The energy required to break these intermolecular solute-solvent hydrogen bonds can be significant and lead to a major transport barrier. However, if such a polar solute molecule is capable of forming strong intramolecular hydrogen bonds, in place of the solute-water hydrogen bonds, then the energy barrier for the transport of the solute across a lipophilic cell membrane will be reduced. In addition, strong intramolecular solute hydrogen bonding will minimize the hydrogen bonding/electrostatic binding of the solute to the polar head groups of the phospholipids that can also inhibit solute permeation.

χ3 is one of the topological indices developed to encode both molecular size and shape information within a common measure. Caco-2 cell permeability is negatively correlated to χ3 in the 6-term model. Thus, the form of χ3 in the 6-term model suggests that the more bulky/large is a solute molecule, the less will be its permeability through a Caco-2 cell membrane which makes intuitive sense. Still, it should be kept in mind that χ3 contributes little to the prediction of the Caco-2 permeation coefficient in the 6-term scoring function, since only three compounds have non-zero χ3 values. χ3 may be a marginal descriptor in terms of significance for this particular the training set.

The previous MI-QSAR studies of eye irritation (see Kulkami et al. (2001), Toxicology Sciences 59:335-45, and Kulkarni and Hopfinger (1999), Pharmaceutical Research 16:1244-52 led to QSAR models which can be mechanistically interpreted as consisting of two contributing factors;

1. AQUEOUS SOLUBILITY—A parabolic relationship is found between eye irritation potency, MES, and aqueous solubility of the solute irritant. In practice, most eye irritants have aqueous solvation free energies, F(H2O), in a range which display a direct linear relationship (half of the parabola ) to eye irritation potency measures.

2. MEMBRANE-SOLUTE INTERACTION/BINDING—A linear relationship is found between increasing (favorable) binding energy of the solute to the phospholipid-rich regions of a membrane and the magnitude of its corresponding MES measures.

These same two factors also appear to partially govern Caco-2 cell permeation, but both contributions exhibit opposite relationships to Pcaco-2 measures as compared to MES measures. An increase in aqueous solubility, as measured by an increasingly negative value of F(H2O), decreases Pcaco-2. The less favorably the solute interacts with the membrane, and/or water, as measured by ΔETT(hbd), ESS(hb) and X3, the larger is the Pcaco-2 measure. But overall, the same two factors that govern the eye irritation potency of a solute may, in fact, also play significant roles in its cellular permeation behavior.

There is an additional factor that appears to be important in governing solute permeability that is not found in the eye irritation MI-QSAR models. The greater the conformational flexibility of the solute within the membrane, the greater the permeability of the solute. In the case of the Pcaco-2 values of the training and test set compounds, conformational flexibility is expressed in the MI-QSAR models mainly by ETT(14) and ETT(tor), as well as by ΔETT(hb) and ESS(hb).

If the six terms in six term scoring function are grouped together in the following manner,

Pcaco-2=−40.50+┌0.65F(H2O)┐+┌0.06ΔETT(hb)−5.61c3┐+┌−0.19ESS(hb)+0.10ETT(14)−0.03ETT(tor)], (3)

then each of the terms within the three sets of bold brackets “define” a contribution to the inferred general mechanism of Caco-2 cell permeation. Hence, eq.(3) can be generalized to the form;

Pcaco-2=(a constant value)−[aqueous solubility]−[membrane-solute binding]+[conformational flexibility of the solute in the membrane] (4)

An important strength of the MI-QSAR approach is to be able to construct simple and statistically significant relationships like the 2- through 6-term scoring functions, and a corresponding general mechanistic equation like equation (4). That is, MI-QSAR analysis is able to generate meaningful ADME property models employing a limited number of descriptors that can be directly interpreted in terms of physically reasonable mechanisms of action. There is no need to resort to generating very large numbers of intramolecular solute descriptors, and then producing a model that meets the statistical constraints of acceptance by performing some type of data reduction.

A number of embodiments of the invention have been described. Nevertheless, it will be understood that various modifications may be made without departing from the spirit and scope of the invention. Accordingly, other embodiments are within the scope of the following claims.

What is claimed is:

1. A method of constructing a modular computational model for predicting one or more therapeutic properties of a chemical compound, comprising:
obtaining a first set of data describing the interaction between each training compound of a first set of training compounds and a first interaction partner; and
using the first set of data, along with data about the chemical structures and/or physical properties thereof of the first set of training compounds and, optionally, data about the three dimensional structure and/or physical properties thereof of the first interaction partner, to construct a first module that uses data about the chemical structures and/or physical properties thereof of chemical compounds to predict values describing the interaction between a chemical compound and the first interaction partner, wherein the predicted values are the same type of data as the data contained in the first set of data;
thereby constructing a single module modular computational model for predicting one or more therapeutic properties of a chemical compound.

2. The method of claim 1, wherein the first set of data is obtained experimentally using a high throughput instrument.

3. The method of claim 2, wherein the high throughput instrument is a multi-channel or multi-cell calorimeter.

4. The method of claim 1, wherein the first set of data includes measurements of enthalpy, ΔH.

5. The method of claim 4, wherein the first set of data includes distinct measurements of enthalpy, ΔH, entropy, ΔS, and free energy, ΔG.

6. The method of claim 1, further comprising:
obtaining a second set of data describing the interaction between each training compound of a second set of training compounds and a second interaction partner;
using the second set of data, along with data about the chemical structures and/or physical properties thereof of the second set of training compounds and, optionally, data about the three dimensional structure and/or physical properties thereof of the second interaction partner, to construct a second module that uses data about the chemical structures and/or physical properties thereof of chemical compounds to predict values describing the interaction between a chemical compound and the second interaction partner, wherein the predicted values are of the same type of data as the data contained in the second set of data;
thereby constructing a two module modular computational model for predicting one or more therapeutic properties of a chemical compound.

7. The method of claim 6, wherein at least one of the modules predicts therapeutic property values that are relevant to the therapeutic potency of compounds.

8. The method of claim 7, wherein the module that predicts values relevant to therapeutic potency is a 4D-QSAR model.

9. The method of claim 7, wherein the interaction partner of the module that predicts values relevant to therapeutic potency comprises a protein.

10. The method of claim 9, wherein the protein is a hormone.

11. The method of claim 6, wherein at least one the modules predicts therapeutic property values that are relevant to one or more ADMET properties of compounds.

12. The method of claim 11, wherein the module that predicts therapeutic values relevant to one or more ADMET properties of compounds is a MI-QSAR model.

13. The method of claim 11, wherein the interaction partner of the module that predicts therapeutic property values that are relevant to one or more ADMET properties of compounds comprises a membrane having properties identical or consistent with biological membranes.

14. The method of claim 13, wherein the membrane is part of a Caco-2 cell.

15. The method of claim 6, wherein at least one of the modules predicts therapeutic property values that are relevant to the therapeutic potency of compounds, and wherein at least one the modules predicts therapeutic property values that are relevant to one or more ADMET properties of compounds.

16. The method of claim 6, further comprising:
obtaining a third set of data describing the interaction between each training compound of a third set of training compounds and a third interaction partner;
using the third set of data, along with data about the chemical structures and/or physical properties thereof of the third set of training compounds and, optionally, data about the three dimensional structure and/or physical properties thereof of the third interaction partner, to construct a third module that uses data about the chemical structures and/or physical properties thereof of chemical compounds to predict values describing the interaction between a chemical compound and the third interaction partner, wherein the predicted values are of the same type of data as the data contained in the second set of data;
thereby constructing a three module modular computational model for predicting one or more therapeutic properties of a chemical compound.

17. The method of claim 16, wherein at least one of the wherein at least one of the modules predicts therapeutic property values that are relevant to the therapeutic potency of compounds, wherein at least one of the other the modules predicts therapeutic property values that are relevant to one or more ADMET properties of compounds, and wherein the final module predicts therapeutic property values distinct form the therapeutic property predictions of the other two modules.

18. A method of evaluating a plurality of test structures for one or more therapeutic properties, comprising:
a) providing a first modular computational model;
b) providing the chemical structure and/or physical properties thereof for all or a part of each member of the plurality of test structures;
c) applying the first modular computational model to each member of the plurality of test structures to obtain a first set of predicted values describing the interaction between each member of the plurality of test structures and one or more interaction partners; and optionally analyzing the values by:
d) comparing the predicted values from the first set of predicted values with one or more reference values; or
e) ranking the predicted values from the first set of predicted values,
thereby evaluating one or more therapeutic properties of the plurality of test structures.

19. The method of claim 18, wherein the first modular computational model includes at least two modules.

20. The method of claim 18, wherein at least one of the modules of the modular computational model makes predictions relevant to the therapeutic potency of test structures.

21. The method of claim 20, wherein the module that makes predictions relevant to the therapeutic potency of test structures is a 4D-QSAR model.

22. The method of claim 21, wherein the 4D-QSAR model predicts enthalpy (ΔH) values.

23. The method of claim 18, wherein at least one of the modules of the modular computational model makes predictions relevant to one or more ADMET properties of compounds.

24. The method of claim 23, wherein the module that makes predictions relevant to one or more ADMET properties is a MI-QSAR model.

25. The method of claim 18, wherein at least one of the modules predicts therapeutic property values that are relevant to the therapeutic potency of compounds, and wherein at least one the modules predicts therapeutic property values that are relevant to one or more ADMET properties of compounds.

26. The method of claim 25, wherein the plurality of test structures is ranked with respect to both therapeutic potency and one or more ADMET properties.

27. The method of claim 26, wherein a subset of the test structures are identified as having a high rank with respect to both therapeutic potency and one or more desirable ADMET properties.

28. The method of claim 18 further comprising a computer readable record of at least some of the pharmaceutical properties predictions generated by evaluation of the plurality of test structures.
